(19) United States
(12) Patent Application Publication
(10) Pub. No.: US 2022/0001192 A1
(43) Pub. Date: Jan. 6, 2022

(54) WEARABLE QUANTUM TREATMENT DEVICE

(71) Applicant: UNHAE ENC CO., LTD., Siheung-si (KR)

(72) Inventor: Boo Yeol KIM, Ansan-si (KR)

(73) Assignee: UNHAE ENC CO., LTD., Siheung-si (KR)

(21) Appl. No.: 17/083,176

(22) Filed: Oct. 28, 2020

(30) Foreign Application Priority Data
Jul. 1, 2020 (KR) 10-2020-0080962

(51) Int. Cl. A61N 2/02; D06M 23/10; D06M 10/10; D06B 1/02; A41D 1/00; A61N 2/00

(52) U.S. Cl. CPC A61N 2/02; D06M 23/10; D06M 10/10; A41D 2400/32; A41D 1/002; A61N 2/002; A61N 2/004; D06B 1/02

(57) ABSTRACT

A wearable quantum treatment device embodied in a garment made in such ways as making a conductive fabric into a garment for a human body; sewing a common fabric with quantum energy generating coils; sewing a fabric of a conductive film with quantum energy generating coils; winding a metal wire into quantum energy generating coils and attaching the same to a fabric, such that a power supply unit disposed at one side of a lower portion of the front or the back of a garment supplies a variable DC or AC power in a form of pulsed electromagnetic field to the quantum energy generating coils, whereby pulsed electromagnetic fields are generated in directions opposite to each other, thereby overlapping and dissipating each other so as to create and irradiate quantum energy in a state of zero strength electromagnetic field to a human body, thus eventually improving health and treating diseases.

### CROSS-REFERENCE TO RELATED APPLICATIONS

This application claims under 35 U.S.C. § 119(a) the benefit of priority to Korean Patent Application No. 10-2020-0080962 filed on Jul. 1, 2020, the entire contents of which are incorporated herein by reference.

### TECHNICAL FIELD

The present disclosure relates to a wearable quantum treatment device, more particularly, a device integrated with clothes such as a hat, a top, a glove, a pair of trousers, a pair of socks, etc by weaving a garment with a conductive fabric, partially spraying a conductive material, through a perforated frame having a form of a quantum energy generating coil, onto a normal fabric, attaching the device to a fabric of a flexible printed circuit board having a form of a quantum energy generating coil, laser cutting a conductive metal sheet into a form of a quantum energy generating coil to insert or stack the same between an upper fabric and a lower fabric and then sewing them together, or winding a conductive metal wire or a sheathed electric wire over a base fabric to make a quantum energy generating coil, which is then stacked and sewed with a covering fabric, wherein a power supplier, which may be installed inside a pouch or a pocket formed at a front lower part or a rear lower part of an upper garment, provides a power as a form of a pulsed electromagnetic field to first and second quantum energy generating coils 360a, 360b, which then generate variable pulsed electromagnetic fields in two opposing directions, both being perpendicular to a direction of a current, whereby while overlapping and dissipating each other the electromagnetic fields create a state of zero strength electromagnetic field in which quantum energy is irradiated to a human body, thereby improving health and treating diseases.

### BACKGROUND

Recently, wearable electric devices having such features as input/output, operation, and telecommunication have been developed for use in close proximity to, or in contact with, a human body. Wearable electric devices may include such accessory type devices as a wrist watch, glasses, earpieces as well as wearable smart devices integrated with a textile to form a garment having electric functions. And such wearable smart devices as a body type in close contact with a human body, a skin patch type, an implantable type to be implanted inside a human body have also been developed.

The quantum energy generator (QEG), as invented by Nikola Tesla, comprises two cylinders without metal cores, in which two cylinders wound, one to the left and the other to the right, by wires, to which electric power is applied, thereby creating electromagnetic fields in directions perpendicular to the direction of current, wherein the two electromagnetic fields, being opposed to each other due to the opposing winding directions of the coils, overlap and counteract each other to make the strength thereof zero (o), when scientists found emergence of a new energy and called this non hertzian waves, quantum energy or scalar energy.

Möbius coil, which is named after a German mathematician, August Ferdinand Möbius, is a coil which modifies Tesla coil such that coils are spooled, in a twisted infinite loop, around cylinders without metal cores with currents flowing in opposing directions; thus, unlike Tesla coil wherein a generated quantum energy field has a frequency identical to that of applied current, Möbius coil as improved generates a quantum energy field of a specific frequency represented by a group of harmonic functions compensated through amplification, offsetting, and interference, etc. (Tesla coil is a precursor of the quantum energy generating device).

Wave patterns of human bodies According to the quantum theory of David Bohm, the father of modern quantum medicine, each entity of molecules, cells, tissues, and organs which constitute living things including human bodies has a dual structure of particle structure and wave structure (quantum potential) or quantum wave field.

That is, each entity of molecules, cells, tissues, and organs has its own wave field.

Quantum wave fields of molecules, cells, tissues, organs, and entities are called molecular field, cellular field, tissue field, organ field, and entity field, respectively.

On the quantum wave field of the molecules of water which constitutes human bodies, Emilio Del Giudice at the Italian Institute for Nuclear Physics (INFN) suggested that water can be categorized into two types: water in a non-coherent state and water in coherent domains. Giudice suggested that waster in coherent domains have properties of a quantum wave field.

He further stated that when water is in a quantum wave field its molecules have electrostatic attraction forces, thereby causing interference even at a distance, reducing the hydrogen bond between electric dipoles while increasing the level of coherence as well as decreasing the size of water clusters leading to a “micro-cluster” phenomenon in a permanently stabilized ground state.

He argued that applying a small electric disturbance in water can cause electric polarization, thereby leading to a quantum wave field of water. Herein, coherence is a physics term which means that two electric dipoles spaced apart from each other fluctuate while affecting each other.

Del Giudice found that water covering and interfacing with a cell membrane of a human body, as observed by the nuclear magnetic resonance, has a structure distinct from that of water spaced apart from the membrane, so he called this “water of microclusters”.

A German scientist Fritz-Albert Popp announced the results of his research as follows.

DNA in a living cell stores and releases biophotons. The biophotons released from DNAs of living cells is a staunch communication means between DNAs, and the biophotons as Popp defined mean a quantum wave field of DNAs with a high degree of coherence.

Herein, coherence is a physics term which means that two electric dipoles spaced apart from each other fluctuate while affecting each other.

### Quantum Field of Organs

In 1991, Rollin McCraty at the HeartMath Institute in the U.S. published the results of his research which indicate that the heart is surrounded by “a weak energy field” shaped like a donut extending outside the body. “The weak energy field” herein means a quantum wave field.

### Quantum Field of Brain

In 1990, Demetri Psaltis reported on the quantum wave field of the brain. He suggested that the brain is not just a physical organ but involves a quantum wave field; every part of the brain is non-locally integrated as an entity just like a hologram. And the quantum wave field of the brain is non-linear and has a high degree of coherence.

### Quantum Field of Entity

Barbara Brennan, a former researcher for atmospheric physics at NASA, suggested that a human body is surrounded by its own energy field, “Aura” as she called. Her research on Aura, in summary, indicates that numerous organisms including from fungi to plants and from frogs to human beings have their own quantum wave fields surrounding themselves; a healthy organism has a different quantum wave field from that of a sick one; a quantum wave field of an organism changes before the organism gets sick.

### Research on Qi

Qi is a kind of individual energy fields. The oriental medicine traditionally assumes that a human body has its own quantum wave field, called “Qi” which flows through a network of channels called “meridian system” along which there are a number of unique points where acupuncture is performed for treatment.

As discussed, the quantum wave field or quantum energy plays a critical role in improving health of human bodies as well as in treating diseases.

Korean Patent Laid-open Gazette No. 10-1435008 (titled “Wearable Computer and Method of Interconnecting Conducting Fiber and Electronic Module of the Wearable Computer”) discloses a wearable computer wherein there is provided a wearable computer including an electronic module, comprising: a metal wire connected to the electronic module and having a circular ring portion on the electronic module side; and conductive fibers which are arranged in a spiral shape alternately arranged spirally with the metal wire from a predetermined point of the circular ring, thereby giving an advantage that the metal wire and the conductive fiber can be stably bonded and damage to the connection portion can be prevented; however, even with such similarities as using conductive fibers, electronic module attached to garments, etc. to the present invention, the disclosure does not involve a feature of treating human bodies by forming quantum coils to generate and superposing electromagnetic fields so as to obtain quantum energy for treatment.

Korean Patent Laid-open Gazette No. 10-2087267 (titled “An App-driven Type Miniaturized Wearable Electrotherapy Device for Reduction of Exercise Pain”) discloses an app-driven wearable electrotherapy device, comprising a power supply part 13 for converting a power supply of the provided battery into a set electrical pulse signal; a switching unit 14 for applying the pulse signal in a circulating manner in the electrode unit selected at a predetermined period through the expansion unit; an expansion connector 15 connected to the switching unit 14 to output a pulse signal; a first communication unit transmitting an operation information including the connection and operation state information of the auxiliary pad connected to the power supply and the switching unit; an auxiliary pad having an auxiliary electrode 161 connected to the expansion connector 15 and outputting the applied pulse signal to the lower side; a first storage unit storing a control signal including a strength and a period of a pulse signal; an interface unit 25 that receives and selects a control signal of the first storage unit 22 and outputs motion information; a second storage unit which stores image information including body parts, muscles, and joints, pain information corresponding to the image information, and attachment position information of the main pad and the auxiliary pad corresponding to the pain information; a support unit configured to present image information and pain information of the storage unit through the interface unit, and provide attachment position information corresponding to the selected pain information; a control unit 26 transmitting a control signal selected by the user with the interface unit 25 through the second communication unit 21 and outputting the received operation signal through the interface unit 25; characterized by a feature of reducing pain by applying a pulse signal reflecting a bodily pain information of a subject to auxiliary electrodes and electrodes attached, respectively, to an auxiliary pad and a main pad covering a part of the body of the subject; thus lacking a feature of generating quantum energy.

Korean Patent Laid-open Gazette No. 10-2091512 (titled “Apparatus for Emitting Quantum Energy for Treatment and Health Management”) discloses apparatus for Emitting Quantum Energy for Treatment and Health Management, comprising a nitrogen oxide supply comprising a first nitrogen oxide supply and a second nitrogen oxide supply; a first quantum energy generator comprising a first quantum energy generator and a second quantum energy generator, a first microcurrent supply for activating the intracellular ion channel and removing the pain-causing factors to remove the pain by neutralizing the cations accumulated in the pain-induced area by flowing negative electricity while injecting nitrogen oxide and hydroxyl ion (OH-Radical) ions and irradiating quantum energy to the body's treatment and health care areas; a second quantum energy generator comprising a third quantum energy generator and a fourth quantum energy generator, a second microcurrent supply device that activates intracellular ion channels and eliminates pain-causing factors by sending negative electricity while investigating the quantum energy generated by the quantum energy generator at the pain causing point and neutralizing the cations accumulated at the pain causing point; a control panel for supplying power to the first quantum energy generator and the second quantum energy generator, wherein during a high voltage discharge nitrogen and oxygen molecules, both of which constitute air, are dissociated to produce nitrogen and oxygen atoms, and then nitric oxides by ionic bonds, which, in turn, are injected onto the patient's pain induced area, where at the same time the cations accumulated are neutralized by flowing negative electricity, thereby being capable of irradiating quantum energy to local pain areas, but still lacking the capability of radiating quantum energy to an entire body or a selected part of the body.

That is, the existing technology for medical treatment by generating and irradiating quantum energy with wearable devices has the disadvantages discussed above; thus there is still a need for developing a novel technology for generating and irradiating quantum energy with broader applications as well as feasibility, stability, and durability, thereby improving health and treatment of diseases without causing inconvenience in everyday life either at home or at work.

### PRIOR ART DOCUMENTS

### Patent Documents

(Patent Document 0001) Korean Patent Laid-open Gazette No. 10-1435008

(Patent Document 0002) Korean Patent Laid-open Gazette No. 10-2087267

(Patent Document 0003) Korean Patent Laid-open Gazette No. 10-2091512

### SUMMARY

### Problems to be Solved

The present disclosure is directed to a wearable quantum treatment device, wherein electric power, supplied from a power supply unit selected from a group consisting of a first power supply unit, a second power supply unit, a third power supply unit, a fourth power supply unit, and a fifth power supply unit, is applied to a reverse polarity converter, which, according to a control circuit programmed and input in advance, supplies power to quantum energy generating coils in such a way of periodically reversing a polarity of a direct current being applied thereto; the quantum energy generating coils are made of any one of a group of materials consisting of a first material which is a conductive fabric, a second material which is a generic porous fabric, onto which a conductive material is partially sprayed through a frame shaped like a quantum energy generating coil thereby imparting conductivity thereto, a third material which is a flexible printed circuit board made by first spreading a resin composition onto a base film and then stacking a conductive metal sheet machined in a shape of a quantum energy generating coil and hot forming both together, a fourth material comprising an upper fabric and a lower fabric between which a conductive metal sheet laser-cut into a shape of a quantum energy generating coil is inserted, stacked, and sewed up together; a fifth material made by disposing a coil of a metal wire of predetermined diameter insulated or a sheathed electric line as wounded in a shape of a quantum energy generating coil in contact with a base fabric and then stacking, thereonto, a covering fabric and then sewing up them together; a quantum energy generating coil is embodied in any one of a group consisting of a first form in which a coil is wound from top to bottom, with intervals, forming a zigzag pattern, a second form in which a coil is wound, laterally, with intervals, forming a zigzag pattern, a third form which is a solenoid coil transformed into a shape of disc; a fourth form of toroid coil, a fifth form of Möbius coil, a sixth form of Helmholtz coil; a seventh form (not shown) of a combination thereof; the device, as made of any one of the materials and embodied in any one of the forms, is integrated with clothes such as a hat, a top, a pair of gloves, a pair of trousers, a pair of socks, etc. by weaving a garment with a conductive fabric, partially spraying a conductive material, through a perforated frame having a form of a quantum energy generating coil, onto a normal fabric, attaching the device to a fabric of a flexible printed circuit board having a form of a quantum energy generating coil, laser cutting a conductive metal sheet into a form of a quantum energy generating coil to insert or stack the same between an upper fabric and a lower fabric and then sewing them together, or winding a conductive metal wire or a sheathed electric wire over a base fabric to make a quantum energy generating coil, which is then stacked and sewed up with a covering fabric, wherein a power supply, which may be installed inside a pouch or a pocket formed at a front lower part or a rear lower part of an upper garment, provides a power as a form of a pulsed electromagnetic field to first and second quantum energy generating coils 360a, 360b, which then generate variable pulsed electromagnetic fields in two opposing directions, both being perpendicular to a direction of current, whereby while overlapping and dissipating each other the electromagnetic fields create a zero strength electromagnetic field state in which quantum energy is irradiated to a human body, thereby improving health and treating diseases.

### Method for Solving the Problem

To achieve the objective discussed above, the wearable quantum treatment device of the present disclosure comprises: a power supply 100 selected from a group of power supply units consisting of a first power supply unit 110 having a battery cell 111, a DC/DC converter 112, a control unit 113 with such integrated control capabilities as a voltage determining unit 113a, a voltage/current detecting sensor 113b, a control circuit 113c, a power cut-off switch 113d, a temperature sensor (not shown) detecting surface temperature of the quantum energy generating coils 360a, 360b as well as surface temperature of a body part where the device is applied, thereby supplying a variable electric power as a form of a pulsed electromagnetic field to the first and second quantum energy generating coils 360a, 360b;

a second power supply unit having an step-down transformer 121, a control unit 123 with such integrated control capabilities as a rectifier circuit 123a, an operating module 123b, a control module 123c, a temperature sensor (not shown) detecting surface temperature of the quantum energy generating coils 360a, 360b as well as surface temperature of a body part where the device is applied, and a current detecting sensor 124, thereby supplying a variable electric power as a form of a pulsed electromagnetic field to the first and second quantum energy generating coils 360a, 360b;

a third power supply unit 130 having an AC power generating unit 131 with a first electrode 131a, a piezoelectric material 131b, a second electrode 131c, and a conductive wire 131d integrated, an AC/DC converter 132, a DC/DC converter 133, a control unit 134 with control capabilities having a temperature sensor (not shown) detecting surface temperature of quantum energy generating coils 360a, 360b as well as surface temperature of a body part where the device is applied, and a current detecting sensor 134, thereby supplying a variable electric power as a form of a pulsed electromagnetic field to the first and second quantum energy generating coils 360a, 360b;

a fourth power supply unit 140 having a rectifier 141, a converter 142, an inverter 143, a resonant reactor 144, a pulse transformer 145, a control unit 146 with such integrated control capabilities as a temperature sensor (not shown) detecting surface temperature of the quantum energy generating coils 360a, 360b as well as surface temperature of a body part where the device is applied, a gate actuating unit 147, a first condenser 148a, and a second condenser 148b, thereby supplying a variable electric power as a form of a pulsed electromagnetic field to the first and second quantum energy generating coils 360a, 360b;

a fifth power supply unit 150 having a power supply 151 of either of an AC power supply 151a and a DC power supply 151b, an AC/DC converter 152, an AST (Automatic Transfer Switch) 153, a low frequency output unit 154, a switching device 155, a control unit 156 with such control capabilities as a temperature sensor (not shown) detecting surface temperature of the quantum energy generating coils 360a, 360b as well as surface temperature of a body part where the device is applied, thereby supplying a variable electric power as a form of a pulsed electromagnetic field to the first and second quantum energy generating coils 360a, 360b;

a reverse polarity converter, wherein a variable (current or voltage) DC power from a power supply 100 selected from a group consisting of a first power supply unit 100, a second power supply unit 120, a third power supply unit 130, a fourth power supply unit 140, a fifth power supply unit 150, is provided first, via a positive conductive wire 201 and a negative conductive wire 202, to a first uncontrollable switch 211 and input wires 212a-1, 212d-1 of a second switch 212 controlled by a micro controller, by a control of which a contact 3 212c of the second switch comes into contact with a contact 2 212b, and a contact 1 212a comes into contact with a contact 4 212d, thereby reversing the polarities (that is, a negative becomes a positive, and vice versa), whereby a variable power is applied to the first and second quantum energy generating coils 360a, 360b via output wires 212b-2, 212d-2 of the second switch;

materials of the quantum energy generating coils made by using one or more selected from a group consisting of: a first material configuration, as made by weaving a conductive fabric, having a porous fabric structure 311, pores 312 of the porous fabric structure, a conductive material layer 313 filling the pores 312, a resin layer 314 coating an outer surface of the conductive material layer;

a second material configuration made by first spreading a fabric 321 of a porous fabric structure on a working table 322, and then arranging a frame 323 perforated in a form of a quantum energy generating coil selected from a group consisting of the first form 351 to the 6th form 356 or a seventh form 357 as combinations thereof, in contact with the fabric prepared, and then spraying, with a sprayer prepared (not shown), a conductive material thereonto, thereby filling the conductive material layer 321b into the pores 321a of the porous fabric structure and then drying the same at a temperature ranging from 80 to 120° C. for 1 to 2 hours;

a third material configuration made by a process comprising: preparing a base film 331; supplying a first resin to a reactor (not shown) 332a; supplying a second resin 332b; providing an inorganic filler 332c; adding 332d and stirring 332e a hardening medium, thereby producing a resin composition; spreading the resin composition 332; stacking quantum coils 333; hot-forming 334; hardening 335, thereby producing a flexible printed circuit board;

a fourth material configuration made by laser-cutting a metal disc made of any one selected from a group consisting of copper (CU), stainless steel (ST304, STS316L), silver (Ag), titanium (Ti) in one selected from a group consisting of the first form 351 to the sixth form 356 or a seventh form 357 as a combination thereof to make a quantum energy generating coil 342, which is then inserted and sewed up together;

a fifth material configuration 350 made by using quantum coils of a wire made of any one selected from a group consisting of copper, nickel, etc., or a sheathed wire; and then disposing the quantum coils, wound 350b in any one of the first form 351 to the sixth form 356 or a seventh form as a combination thereof, in contact with a base fabric prepared in advance 350c, onto which a covering fabric separately prepared is stacked and sewed up together 350e to finish the assembly of the quantum energy generating coils 360a, 360b;

quantum energy generating coils embodied in any one selected from a group consisting of a first form 351 in which a coil is wound from top to bottom, with intervals, forming a zigzag pattern, a second form 352 in which a coil is wound, laterally, with intervals, forming a zigzag pattern, a third form 353 which is a solenoid coil transformed into a shape of disc; a fourth form 354 of toroid coil, a fifth form 355 of Möbius coil, a sixth form 356 of Helmholtz coil; a seventh form 357 (not shown) as a combination thereof;

a garment attached, by weaving or sewing, with the quantum energy generating coils, which are disposed on such head covering parts 410 as a cap, a hat, an earflap hat or a helmet, etc; on such upper garments 420 as a jacket, a pique shirt, a shirt with an adjustable neck, a suit, a jumper, etc.; on a pair of gloves for hands 430; on such bottoms 440 covering from the waist to the ankle as a pair of thermal underwear bottoms, a pair of trousers, a skirt, etc.; on a pair of socks for feet such that a winding direction of a quantum energy generating coil at the front of the garment is diametrically opposed to that of said coil at the rear thereof, or a winding direction of said coil at one side is diametrically opposed to that of said coil at the other side.

### Effect of the Present Disclosure

The wearable quantum treatment device of the present disclosure is intended to improve health and treat diseases by irradiating quantum energy to a user regardless of time and space either at home or at work.

And the present disclosure enables a customized health care and treatment by inputting such personal information as an area to be treated and duration of a treatment.

The present disclosure enables time setting, thereby lowering electricity cost and lengthening a battery life.

The present disclosure also enables washing, thereby maintaining cleanliness.

The present disclosure enables improving health and treating diseases by irradiating, to a user's body, quantum energy of a pulsed electromagnetic field in a state of zero strength, which is generated when first and second quantum energy generating coils 360a, 360b create two pulsed electromagnetic fields in two opposing directions, both being perpendicular to a direction of current, which then overlap and offset each other, eventually, to dissipate themselves.

The effects of the present disclosure are not limited to the above-mentioned effects, and other effects not mentioned herein will be clearly understood by those skilled in the art from the following description.

### BRIEF DESCRIPTION OF THE DRAWINGS

The above and other features of the present disclosure will now be described in detail with reference to certain exemplary embodiments thereof illustrated in the accompanying drawings which are given hereinbelow by way of illustration only, and thus are not limitative of the present disclosure, and wherein:

FIG. 1 is a schematic diagram showing an entire configuration of a wearable quantum treatment device of the present disclosure;

FIG. 2 is a sectional view of a first power supply unit, with integrated control capabilities as shown in FIG. 1, which supplies a variable power as a form of a pulsed electromagnetic field to first and second quantum energy generating coils;

FIG. 3a is a sectional view of a second power supply unit, with integrated control capabilities as shown in FIG. 1, which supplies a variable power as a form of a pulsed electromagnetic field to first and second quantum energy generating coils;

FIG. 3b is a sectional view illustrating a third power supply unit, with integrated control capabilities as shown in FIG. 1, which supplies a variable power as a form of a pulsed electromagnetic field to first and second quantum energy generating coils;

FIG. 3c is a sectional view illustrating a fourth power supply unit, with integrated control capabilities as shown in FIG. 1, which supplies a variable power as a form of a pulsed electromagnetic field to first and second quantum energy generating coils;

FIG. 3d is a sectional view of a fifth power supply unit, with integrated control capabilities as shown in FIG. 1, which supplies a variable power as a form of a pulsed electromagnetic field to first and second quantum energy generating coils;

FIG. 4 is a schematic diagram illustrating a straight polarity of a reverse polarity converter as shown FIG. 1;

FIG. 5 is a schematic diagram illustrating a reverse polarity of a reverse polarity converter as shown in FIG. 1;

FIG. 6 is a sectional view of a first material configuration of the first and second quantum energy generating coils of the wearable quantum treatment device as shown in FIG. 1;

FIG. 7a is a section view of a second material configuration of the first and second quantum energy generating coils of the wearable quantum treatment device as shown in FIG. 2;

FIG. 7b is a sectional view of a third material configuration of the first and second quantum energy generating coils of the wearable quantum treatment device as shown in FIG. 1;

FIG. 7c is a sectional view of a fourth material configuration of the first and second quantum energy generating coils of the wearable quantum treatment device as shown in FIG. 1;

FIG. 7d is a section view of a fifth material configuration of the first and second quantum energy generating coils of the wearable quantum treatment device as shown in FIG. 2;

FIG. 8 is a sectional view of a first form of the first and second quantum energy generating coils of the wearable quantum treatment device as shown in FIG. 1;

FIG. 9a is a section view of a second form of the first and second quantum energy generating coils of the wearable quantum treatment device as shown in FIG. 2;

FIG. 9b is a sectional view of a third form of the first and second quantum energy generating coils of the wearable quantum treatment device as shown in FIG. 1;

FIG. 9c is a sectional view of a fourth form of the first and second quantum energy generating coils of the wearable quantum treatment device as shown in FIG. 1;

FIG. 9d is a section view of a fifth form of the first and second quantum energy generating coils of the wearable quantum treatment device as shown in FIG. 2;

FIG. 9e is a sectional view of a sixth form of the first and second quantum energy generating coils of the wearable quantum treatment device as shown in FIG. 1;

FIG. 10 is a sectional view of the head of a user wearing a head cover or a cap integrated with the first and second quantum energy generating coils, as embodied in first, second, third, fourth, fifth, sixth forms and a seventh form as a combination thereof as shown in FIGS. 8, 9a, 9b, 9c, 9d, and 9e, of the wearable quantum treatment device;

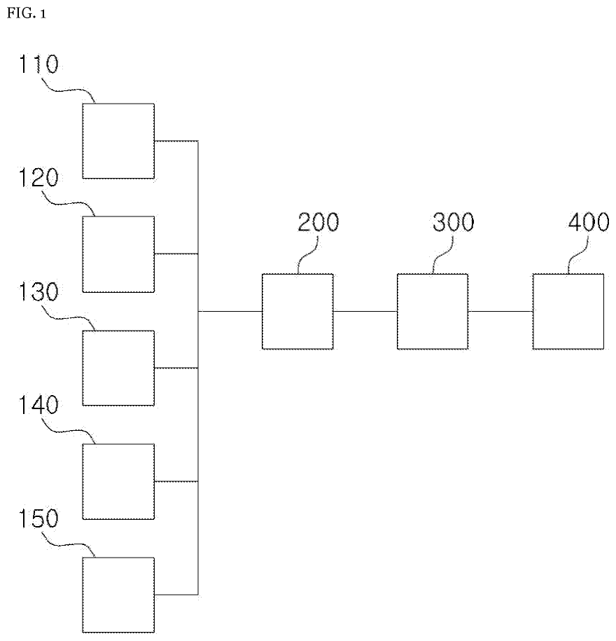

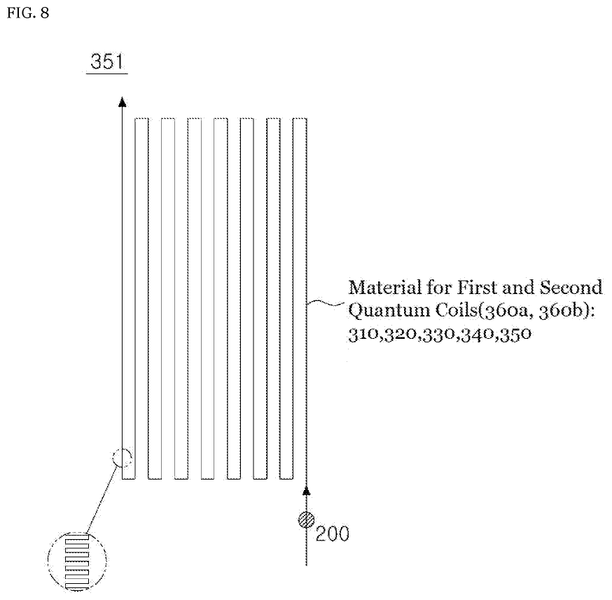

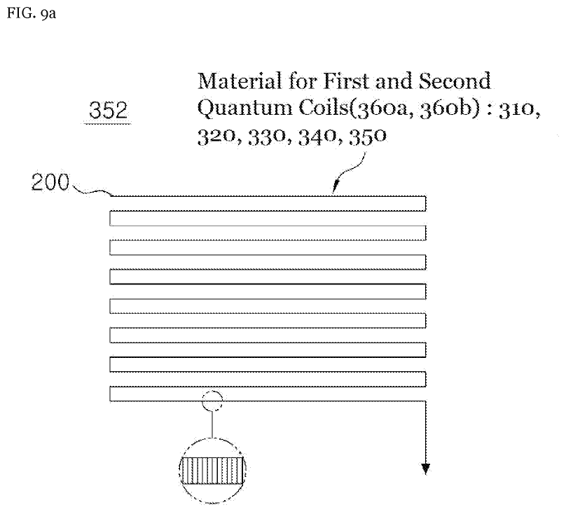

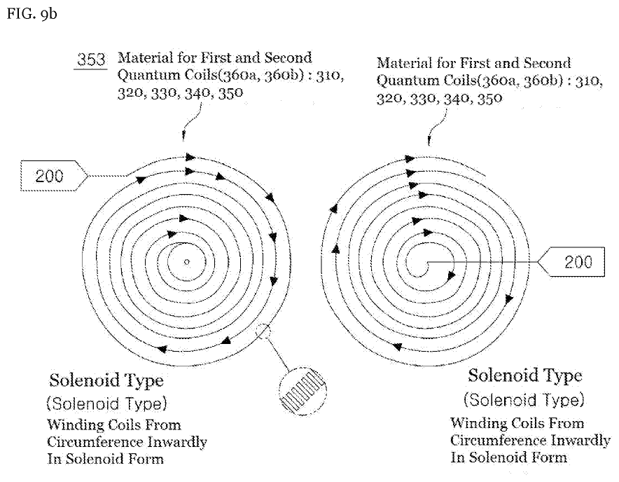

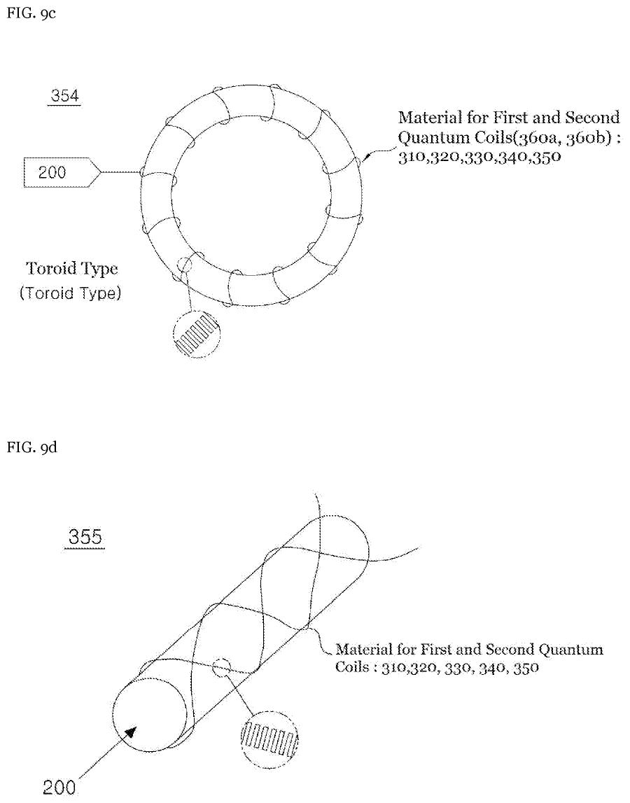

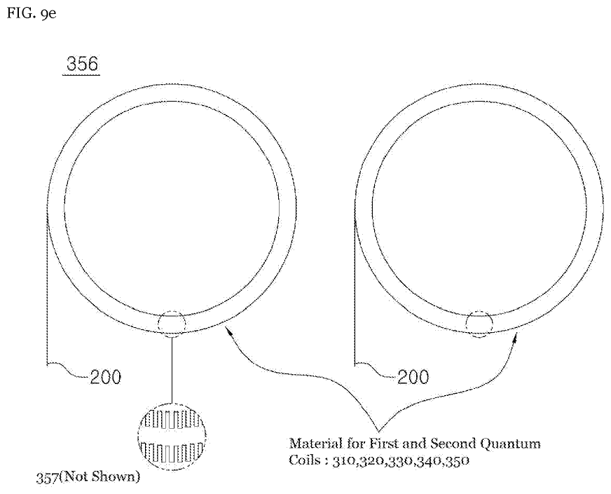

FIG. 1a is a sectional view of upper garments integrated with the first and second quantum energy generating coils, as embodied in first, second, third, fourth, fifth, sixth forms and a seventh form as a combination thereof as shown in FIGS. 8, 9a, 9b, 9c, 9d, and 9e, of the wearable quantum treatment device;

FIG. 11b is a sectional view of pairs of gloves integrated with the first and second quantum energy generating coils, as embodied in first, second, third, fourth, fifth, sixth forms and a seventh form as a combination thereof as shown in FIGS. 8, 9a, 9b, 9c, 9d, and 9e, of the wearable quantum treatment device;

FIG. 1c is a sectional view of pairs of trousers integrated with the first and second quantum energy generating coils, as embodied in first, second, third, fourth, fifth, sixth forms and a seventh form as a combination thereof as shown in FIGS. 8, 9a, 9b, 9c, 9d, and 9e, of the wearable quantum treatment device;

FIG. 11d is a sectional view of pairs of socks or shoes integrated with the first and second quantum energy generating coils, as embodied in first, second, third, fourth, fifth, sixth forms and a seventh form as a combination thereof as shown in FIGS. 8, 9a, 9b, 9c, 9d, and 9e, of the wearable quantum treatment device.

### DETAILED DESCRIPTION OF ILLUSTRATIVE EMBODIMENT

Hereinafter, embodiments of the present disclosure will be described in more detail with reference to the drawings. First, a unique numeral is assigned to each of the elements in figures, and embodiments of the present disclosure will be described in more detail with reference to the numerals of the identical elements. It should be understood that, as a unique numeral is assigned to each of the elements in figures, elements will be always indicated by their unique numerals even when they appear in different figures. And as the present disclosure is described, details of the configuration or function of the prior art relevant to the present disclosure will be omitted when determined that they may possibly obscure the points of the description of the present disclosure. It should be understood that the appended drawings are not necessarily to scale, presenting a somewhat simplified representation of various preferred features illustrative of the basic principles of embodiments of the disclosure. The specific design features of embodiments of the present disclosure as disclosed herein, including, for example, specific dimensions, orientations, locations, and shapes will be determined in part by the particular intended application and use environment.

FIG. 1 is a schematic diagram showing the entire configuration of a wearable quantum treatment device of the present disclosure.

With reference to FIG. 1, the wearable quantum treatment device of the present disclosure comprises: a power supply unit 100 selected from a group consisting of a first power supply unit 110 having a battery cell 111, a DC/DC converter 112, a control unit 113 with such control capabilities as a voltage determining unit 113a, a voltage/current detecting sensor 113b, a control circuit 113c, a power cut-off switch 113d, a temperature sensor detecting surface temperature of the quantum energy generating coils 360a, 360b as well as surface temperature of a body part where the device is applied, thereby supplying a variable electric power as a form of a pulsed electromagnetic field to the first and second quantum energy generating coils 360a, 360b;

a second power supply unit 120 having a step-down transformer 121, a rectifier circuit 122, a control unit 123 with such control capabilities as an input module 123a, an operation module 123b, a control module 123c, a temperature sensor (not shown) detecting surface temperature of the quantum energy generating coils 360a, 360b as well as surface temperature of a body part (the head, torso, hands, lower body, or feet) where the device is applied, and a current detecting sensor 124, thereby supplying a variable electric power as a form of a pulsed electromagnetic field to the first and second quantum energy generating coils 360a, 360b;

a third power supply unit 130 comprising an AC power generating unit 131 having a first electrode 131a, a piezoelectric material 131b, a second electrode 131c, and a conductive wire 131d; an AC/DC converter 132, a DC/DC converter 133, a control unit 134 with such control capabilities as a current detecting sensor 134a, a variable controller 134b, a temperature sensor (not shown) detecting surface temperature of the quantum energy generating coils 360a, 360b as well as surface temperature of a body part (the head, torso, hands, lower body, or feet) where the device is applied, and, thereby supplying a variable electric power as a form of a pulsed electromagnetic field to the first and second quantum energy generating coils 360a, 360b;

a fourth power supply unit 140 having a rectifier 141, a converter 142, an inverter 143, a resonant reactor 144, a pulse transformer 145, a control unit 146 with such control capabilities as a temperature sensor (not shown) detecting surface temperature of the quantum energy generating coils 360a, 360b as well as surface temperature of a body part (the head, torso, hands, lower body, or feet) where the device is applied, a gate actuating unit 147, a first condenser 148a, and a second condenser 148b, thereby supplying a variable electric power as a form of a pulsed electromagnetic field to the first and second quantum energy generating coils 360a, 360b;

a fifth power supply unit 150 having a power supply 151 of either of an AC power supply 151a and a DC power supply 151b, an AC/DC converter 152, an AST (Automatic Transfer Switch) 153, a low frequency output unit 154, a switching device 155, a control unit 156 with control capabilities such as a temperature sensor (not shown) detecting surface temperature of the quantum energy generating coils 360a, 360b as well as surface temperature of a body part (the head, torso, hands, lower body, or feet) where the device is applied, thereby supplying a variable electric power as a form of a pulsed electromagnetic field to the first and second quantum energy generating coils 360a, 360b;

a reverse polarity converter, wherein a variable (current or voltage) DC power from a power supply 100 selected from a group consisting of a first power supply unit 100, a second power supply unit 120, a third power supply unit 130, a fourth power supply unit 140, a fifth power supply unit 150, is provided first, via a positive conductive wire 201 and a negative conductive wire 202, to a first uncontrollable switch 211 and input wires 212a-1, 212d-1 of a second switch 212 controlled by a micro controller, by a control of which a contact 3 212c of the second switch comes into contact with a contact 2 212b, and a contact 1 212a comes into contact with a contact 4 212d, thereby reversing the polarities (that is, a negative becomes a positive, and vice versa), whereby a variable power is applied to the first and second quantum energy generating coils 360a, 360b via output wires 212b-2, 212d-2 of the second switch;

materials of the quantum energy generating coils made by using one or more selected from a group consisting of: a first material configuration, as made by weaving conductive fibers, having a porous fabric structure 311, pores 312 of the porous fabric structure, a conductive material layer 313 filling the pores 312, a resin layer 314 coating an outer surface of the conductive material layer;

a second material configuration made by first spreading a fabric 321 of a porous fabric structure on a working table 322, and then arranging a frame 323 perforated in a form of a quantum energy generating coil selected from a group consisting of the first form 351 to the 6th form 356 or a seventh form 357 as a combination thereof, in contact with the fabric prepared 321, and then spraying, with a sprayer prepared (not shown), a conductive material thereonto, thereby filling the conductive material layer 321b into the pores 321a of the porous fabric structure and then drying the same at a temperature ranging from 80 to 120° C. for 1 to 2 hours;

a third material configuration 331 made by a process comprising: preparing a base film 331; supplying a first resin to a reactor (not shown) 332a; supplying a second resin 332b; providing an inorganic filler 332c; adding 332d and stirring 332e a hardening medium, thereby producing a resin composition; spreading the resin composition 332; stacking quantum coils 333 made in advance in one form selected from a group consisting of the first to seventh forms (not shown); hot-forming 334; hardening 335, thereby producing a flexible printed circuit board;

a fourth material configuration 340 made by laser-cutting a metal disc made of any one selected from a group consisting of copper (CU), stainless steel (ST304, STS316L), silver (Ag), titanium (Ti) into one selected from a group consisting of the first form 351 to the sixth form 356 or a seventh form 357 as a combination thereof to make quantum energy generating coil 343a, 360b, which are then disposed on and in contact with a porous base fabric 341 prepared in advance, and are covered by, and in contact with, a covering fabric, and eventually sewed up together;

a fifth material configuration 350 made by preparing quantum coils 360a, 360b of a wire made of any one selected from a group consisting of copper, nickel, etc., or a sheathed wire; and then disposing 350c the quantum coils, wound 350b in any one selected from a group consisting of the first form 351 to the sixth form 356 or a seventh form 357 as a combination thereof, on and in contact with a base fabric prepared in advance 350a, onto which a covering fabric separately prepared is stacked 350d eventually to finish, by sewing up, 350e the assembly of quantum energy generating coils 360a, 360b;

quantum energy generating coils embodied in any one selected from a group consisting of: forms (351-357) consisting of a first form 351 in which a coil is wound from top to bottom, with intervals, forming a zigzag pattern, a second form 352 in which a coil is wound, laterally, with intervals, forming a zigzag pattern, a third form 353 which is a solenoid coil transformed into a shape of disc; a fourth form 354 of toroid coil, a fifth form 355 of Möbius coil, a sixth form 356 of Helmholtz coil; a seventh form 357 (not shown) as a combination thereof;

a garment attached, by weaving or sewing, with the quantum energy generating coils of any one selected from a group consisting of the first form to the seventh form, which are disposed on such head covering parts 410 as a cap, a hat, an earflap hat or a helmet, etc.; on such upper garments 420 as a jacket covering from the neck to the hip joint, a pique shirt, a shirt with an adjustable neck, a suit, a jumper, etc.; on a pair of gloves for hands 430; on such bottoms 440 covering from the waist to the ankle as a pair of thermal underwear bottoms, a pair of trousers, a skirt, etc.; on a pair of socks for feet;

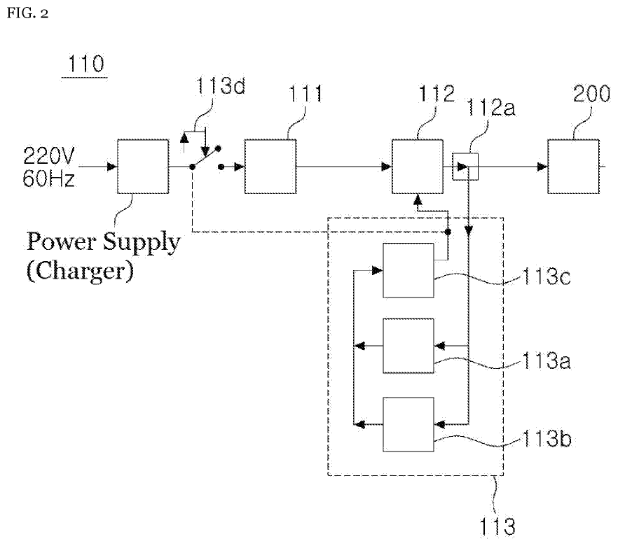

FIG. 2 is a sectional view of a first power supply unit as shown in FIG. 1.

With reference to FIG. 2, the first power supply unit 110 comprises a battery cell 111, a DC/DC converter 112, a control unit 113 with a voltage determining unit 113a, a voltage/current detecting sensor 113b, a control circuit 113c, a power cut-off switch 113d, a temperature sensor (not shown) detecting surface temperature of the quantum energy generating coils 360a, 360b as well as surface temperature of a body part where the device is applied.

The battery cell 111 comprises one or more battery cells with an output of 12 volts, arranged in a series or in parallel.

The DC/DC converter 112 converts power of a predetermined current and voltage received from the battery cell 111 into an output power (4-12 volts) of a variable current and voltage corresponding to a load required by the first and second quantum energy generating coils 360a, 360b and then supplies the same to the reverse polarity converter 200.

The battery cell 111 supplies a DC power to an input terminal (not shown) of the DC/DC converter 112, which automatically turns on a switching device (not shown) for the DC/DC converter, thereby generating an output power, and then turns off the switching device (not shown) when the output power reaches a target level. Once the switching device (not shown) is turned off, the output power decreases until it goes under a reference level, which again turns on the switching device. Repeating turning on and off of the switching device maintains the output power at a steady level.

The control unit 113 comprises a voltage determining unit 113a; a voltage detecting sensor 113b, a control circuit 113c having a pulse width modulation function, a pulse density modulation function, and a pulse repetition rate control function, a power cut-off switch 113d, and a temperature sensor (not shown) detecting surface temperature of the quantum energy generating coils 360a, 360b as well as surface temperature of a body part (the head, torso, hands, lower body or feet) where the device is applied, wherein a voltage detecting sensor disposed at the output side of the DC/DC converter measures a voltage and current of a variable power of a pulsed electromagnetic field, with its form of pulse, pulse period, frequency repetition rate, length of burst frequency, etc. adjusted by the DC/DC converter 112 based on a control signal for pulse width modulation and pulse frequency (density) repetition rate sent from the control circuit 113, supplied to a reverse polarity converter 200, and then transmits, in real time, the measurement to the control circuit 113c of the control unit 113 as well as to the voltage determining unit 113a, which, in turn, compares the measurement with data programmed and input in advance and then transmits any deviation from a standard maintenance value to the control circuit 113c, which, in turn, compares the deviation with an initial input data and transmits a corrected value to the DC/DC/converter 112, which, in turn, supply a variable power of voltage as corrected to satisfy a target value to the reverse polarity converter 200, which, in turn, at a predetermined period, converts the DC power to be supplied to the first and second quantum energy generating coils 360a, 360b into an inverse polarity, that is, a positive to a negative and vice versa, which is eventually supplied to the first and second quantum energy generating coils 360a, 360b. However, when the battery cell 111 is fully discharged or its remaining capacity is below the rated value, thus being incapable of supplying a rated DC power to the DC/DC converter 112, a charging switch disposed between the power supply unit and the better cell 111 is turned on, thereby charging a plurality of the battery cells of the battery cell 111.

And the control unit 113 comprises a separate input interface (not shown) integrated therein, through which a user can provides such information as current, voltage, pulse types, pulse period, burst frequency length as supplied to the first and second quantum energy generating coils 360a, 360b, power supply time and stop time (timer function), functions of the switching device, adjustments of the surface temperature of the first and second quantum energy generating coils 360a, 360b and the body temperature of the user, etc. For the timer function, a user can select, by using the input interface, any one of the sections for power supply period, for example, sections of 30 seconds to 3 minutes, 30 seconds to 10 minutes, 30 seconds to 20 minutes, 30 seconds to 30 minutes, and any one of the sections for stop time, for example, sections of 30 seconds to 3 minutes, 30 seconds to 10 minutes, 30 seconds to 20 minutes, and 30 seconds to 30 minutes.

And a plurality of temperature sensors (not shown) detect temperature of the quantum energy generating coils 360a, 360b integrated with a garment (for example, a hat 410, a top 420, a pair of gloves 430, a bottom 440, a pair of socks 450 or a pair of shoes (not shown)) and temperature of a user's body, and then send the results, via a conductive wire (not shown), to a control unit 113, which then analyzes the results received to control the output power of the power supply unit 110 as a pulsed electromagnetic field to be supplied to the first and second quantum energy generating coils 360a, 360b so as to maintain the temperature at a set level.

With such information as power supply time and downtime input in a way as described above, the device can be used for a longer time even with a limited capacity of the battery charged a single time and irradiate the quantum energy to a human body in a form of pulsed waves.

The battery is one type selected from a group of type of rechargeable (secondary) batteries consisting of a nickel-cadmium (Ni—Cd) battery, an alkaline battery, a nickel metal hybrid battery (Ni-MH), a sealed lead-acid battery (SLA), a lithium-ion battery, and a lithium polymer battery, and is connected to a rectifier circuit which converts alternating current to direct current, which is used for charging the battery.

The first power supply unit 110, as shown in FIG. 1a, is stored in a separate pocket (not shown) made and located, inside or outside, at one side of a lower front part or one side of lower rear part of an upper garment covering a torso (the thorax and belly, etc.).

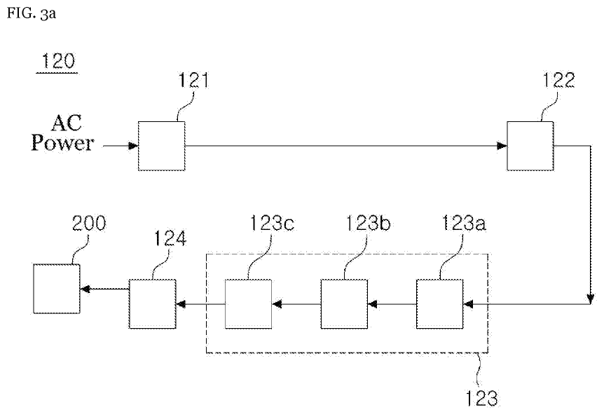

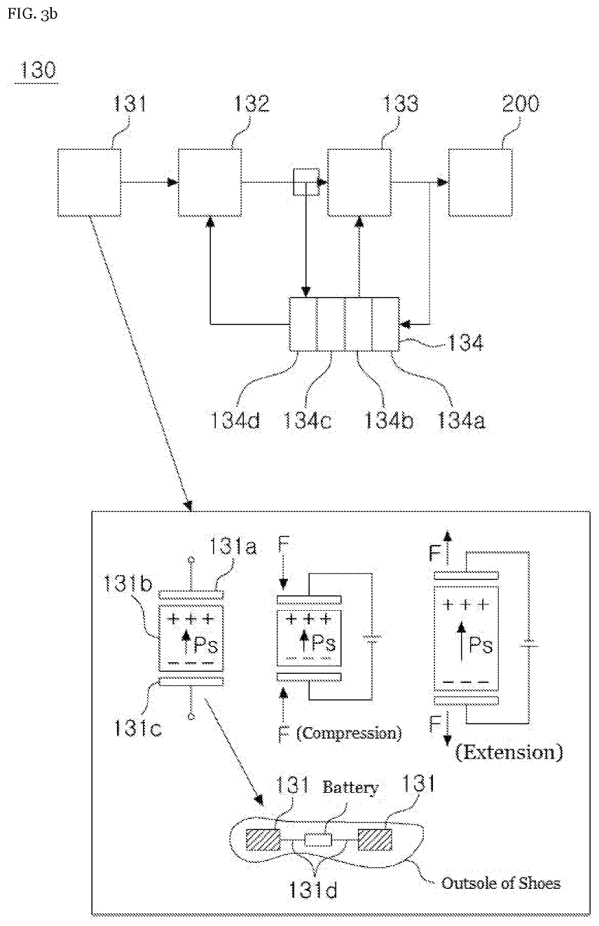

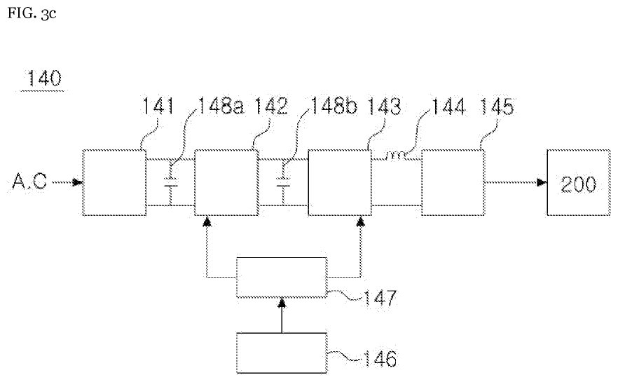

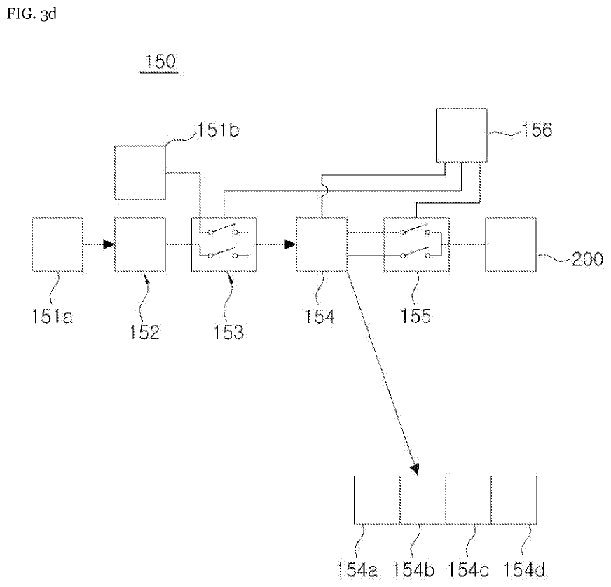

FIG. 3a is a sectional view of the second power supply unit as shown in FIG. 1.

With reference to FIG. 3a, the second power supply unit 120 comprises an step-down transformer 121; a rectifier circuit 122; a control unit 123 with such control capabilities as an input module 123a, an operating module 123b, a control module 123c with a pulse width modulation function, a pulse density modulation function, and a pulse repetition rate control function integrated, a temperature sensor detecting surface temperature of the quantum energy generating coils 360a, 360b as well as surface temperature of a body part (the head, torso, hands, lower body, or feet), wherein a single phase alternating current power of 220 volts and 60 Hz is supplied to the step-down transformer 121, which, in turn, converts the primary voltage to a low secondary voltage ranging from 6 to 50 volts at 60 Hz, which is then converted into a single phase direct current power ranging from 6 to 50 volts by the rectifier circuit 122.

The input module 123a of the control unit 123 is used by a user, via a stored program, to first select any one of operation steps, for example, of steps 1 to 10, and, based on the selected operation step, set such parameters as a voltage (V) of a direct current power, for the selected operation step, supplied, via the reverse polarity converter 200, to the first and second quantum energy generating coils 360a, 360b, more particularly, a minimum value (mA) and a maximum value (A) of a variable current flows through the first and second quantum energy generating coils 360a, 360b, a minimum value (mT) and a maximum value (T) of a variable electromagnetic field, a power supply time for a selected operation step (seconds to minutes or minutes to hours) including a current value, a voltage value, a pulse width, a pulse density period, a burst frequency length, a main power supply time and downtime (timer function), a switching device function, a surface temperature of the quantum energy generating coils 360a, 360b, and a temperature of a body part where the device is applied.

The operation module 123b runs an operation program to calculate, by using a plurality of parameters input, via the input module 123a, by a user, a current value corresponding to a strength of an electromagnetic field to be generated by the first and second quantum energy generating coils 360a, 360b for generating variable quantum energy, wherein a current value is determined in such a way of matching, one by one, strength values of electromagnetic fields with current values based on the relevant data generated in advance. For example, if a strength of an electromagnetic field is to be determined from a range of 1 to 10 steps and a current value is set with the same range, one step of strength of an electromagnetic field generates one step of current value.

The control module 123c generates, based on a current value transmitted from the operation module 123b, a current corresponding to a strength of an electromagnetic field to be generated by the first and second quantum energy generating coils 360a, 360b, and then supplies the same, via the reverse polarity converter 200, to the first and second quantum energy generating coils 360a, 360b, while the current detecting sensor, disposed on one or each of lead-in wires of the first and second quantum energy generating coils 360a, 360b, detects a current flowing on one or each of the lead-in wires and transmits a current value detected, in real time, to the operation module 123a, which compares the current value received with setting values of parameters for a selected operation step, and, in case of the current value deviating from either an upper limit or a lower limit, corrects the deviation so as to transmit normalized setting values of the parameters for a selected operation step to the control module 123c, which, in turn, supplies a power, adjusted based on the normalized setting values of the parameters, to the reverse polarity converter 200, which, in turn, at a time period (for example, a range from 30 seconds to 5 minutes) set in advance, converts the polarity of the power to be supplied to the first and second quantum energy generating coils 360a, 360b, that is, a positive to a negative and vice versa, and then supplies the converted power as a form of a pulsed electromagnetic field to the first and second quantum energy generating coils 360a, 360b.

And the operation module 123b compares a current value generated as above with a current current value received, as digitized, from the first and second quantum energy generating coils 360a, 360b, and transmits a required current value, calculated based on PID, to the control module 123c.

And the quantum energy generating coils 360a, 360b, as integrated with a user's garment (a hat 410, a top 420, a pair of glove 430, a bottom 440, a pair of socks 450, or a pair of shoes (not shown)) transmit, in real time, the information of a temperature of their own as well as of the user's body part (the head, torso, hands, lower body, and feet) where the device is applied, as detected by a plurality of temperature sensors (not shown), via a conductive wire (not shown), to the control unit 123, which, in turn, analyzes the data of the temperature detected and, controls the output power of the power supply unit 120 supplying power to the first and second quantum energy generating coils 360a, 360b so as to maintain the temperature at a set level.

The first and second quantum energy generating coils 360a, 360b are embodied in any one selected from a group consisting of a first form in which a coil is wound from top to bottom, with intervals, forming a zigzag pattern, a second form in which a coil is wound, laterally, with intervals, forming a zigzag pattern, a third form which is a solenoid coil transformed into a shape of disc; a fourth form of toroid coil, a fifth form of Mobius coil, a sixth form of Helmholtz coil, a seventh form (not shown) as a combination thereof.

The second power supply unit 120, as shown in FIG. 11a, is stored in a separate pocket (not shown) made and located, inside or outside, at one side of a lower front part or one side of a lower rear part of an upper garment covering a torso (the thorax and belly, etc.).

FIG. 3b is a sectional view of the third power supply unit as shown in FIG. 1.

With reference to FIG. 3b, the first power supply unit 130 comprises an AC/DC converter 132, a DC/DC converter 133, a control unit 134 with a temperature sensor (not shown) detecting surface temperature of the quantum energy generating coils 360a, 360b as well as of a user's body part (the head, torso, hands, lower body, and feet) where the device is applied, a pulse width modulation function, a pulse frequency modulation function and a pulse frequency (density) control function, and a pulse repetition rate function integrated.

The AC power generating unit 131 comprises a first electrode 131a, a piezoelectric material 131b, a second electrode 131c, a conductive wire 131d.

The first electrode 131a and the second electrode 131c are made of any one selected from a group of materials consisting of titanium (Ti), platinum (Pt), tantalum (Ta), chromium (Cr), iridium (Ir), nickel (Ni), palladium (Pd), copper (Cu), silver (Ag), gold (Au), Iron (Fe); the piezoelectric material 131b is made of any one selected from a group of materials consisting of lead zirconate titanate (PZT), barium titanate (BaTiO3), ammonium dihydrogen phosphate, ethylenediamine tartrate, piezoelectric ceramics made by sintering crystal or barium titanate; a plurality of piezoelectric devices attached under the shoes of a user, wherein the pressure (the weight) created by walking of a user is delivered to the plurality of piezoelectric devices inserted and stacked between a first electrode 131a and a second electrode 131b, thereby deforming the plurality of piezoelectric devices while generating an alternating current power (electromotive power), which is provided, via a conductive wire 131d, to the AC/DC converter 132.

The AC/DC converter 132 converts a general purpose alternating current power to a direct current power, wherein an alternating current power generated and supplied by the AC power generating unit 131, with its electromagnetic interferences removed by a filter (not shown), is rectified and smoothed by a rectifier (not shown), and then corrected by a power factor corrector (not shown) to eventually be supplied to the DC/DC converter 133.

The DC/DC converter 133 converts power received, by adjusting pulse shape, pulse period, frequency repetition rate, burst frequency length, etc. based on control signals from the control unit 134, into a power of a pulsed electromagnetic field, which is output and supplied, via a reverse polarity converter 200, to the first and second quantum energy generating coils 360a, 360b.

The control unit 134 controls switching of the direct current, based on a feedback reflecting changes of output power supplied, via the AC/DC converter 132 and then the reverse polarity converter 200, eventually to the first and second quantum energy generating coils 360a, 360b, so as to vary the level of voltage of the direct current power. And the control unit 134 further comprises a current detector 134a for detecting changes of a load current of the output power supplied, via the reverse polarity converter 200, to the first and second quantum energy generating coils 360a, 360b; a variable controller 134b for generating a first signal and a second signal once the load current supplied from the current detector 134a exceeds a predetermined limit; a first controller 134c for controlling switching of the DC/DC converter based on the first signal from the variable controller 134b; a second controller 134d for controlling switching of a power factor corrector (not shown) based on the second signal from the variable controller 134b.

And the control unit may be further provided with such functions as power supply time and downtime functions (timer function), switching device function, functions of varying the surface temperature of the quantum energy generating coils 360a, 360b as well as a user's body part where the device is applied.

And the quantum energy generating coils 360a, 360b, as integrated with a user's garment (a hat 410, a top 420, a pair of glove 430, a bottom 440, a pair of socks 450, or a pair of shoes (not shown)) transmit, in real time, the information of a temperature of their own as well as of the user's body part (the head, torso, hands, lower body, and feet) where the device is applied, as detected by a plurality of temperature sensors (not shown), via a conductive wire (not shown), to the control unit 135, which, in turn, analyzes the data of the temperature detected and, controls the output power of the power supply unit 130 supplying power to the first and second quantum energy generating coils 360a, 360b so as to maintain the temperature at a set level.

The battery is one type selected from a group of types of rechargeable (secondary) batteries consisting of a nickel-cadmium (Ni—Cd) battery, an alkaline battery, a nickel metal hybrid battery (Ni-MH), a sealed lead-acid battery (SLA), a lithium-ion battery, and a lithium polymer battery, and a plurality of units thereof are provided and installed in a series or in parallel.

The third power supply unit 130, as shown in FIG. 1a, is stored in a separate pocket (not shown) made and located, inside or outside, at one side of a lower front part or one side of a lower rear part of a top covering a torso (the thorax and belly, etc.).

FIG. 3c is a sectional view of the fourth power supply unit as shown in FIG. 1.

With reference to FIG. 3c, the fourth power supply unit 140 comprises a rectifier 141; a converter 142; an inverter 143, a resonant reactor 144; a pulse transformer 145; an AC/DC converter 143, a DC/DC converter 145, a control unit 146 with control capabilities having a temperature sensor (not shown) detecting surface temperature of quantum energy generating coils 360a, 360b as well as surface temperature of the body part (the head, torso, hands, lower body, or feet) where the device is applied; a gate actuating unit 147; a first condenser 148a; and a second condenser 148b.

The rectifier 141 converts an input of a single phase AC power of 220 volts at 60 Hz into a DC power.

The converter 142 steps up voltage from its input DC power, as converted from an AC power by the rectifier 141, to an output.

The inverter 143 modulates a DC power as stepped up by the converter 142, by adjusting pulse shape, pulse period, frequency repetition rate, burst frequency length, etc. based on control signals from the control unit 146, into an AC power as a form of a pulsed electromagnetic field.

The resonant reactor 144 matches loads of the first and second quantum energy generating coils 360a, 360b.

The pulse transformer 145 steps up the output power of the inverter 143. The fourth power supply unit further comprises a control unit 146 which generates a signal to control output power of the converter 142 so as to modulate the switching output power of the first and second quantum energy generating coils 360a, 360b as well as the inverter 143 to which the output power of the pulse transformer 145 is applied, and generates a signal to adjust, regardless of pulse width, the pulse frequency (density) control (PDM) so as to vary the strength of the electromagnetic field generated by the first and second quantum energy generating coils 360a, 360b and eventually to vary the amount of quantum energy to be generated; a gate actuating unit 147 which amplifies the voltage of a control signal from the control unit 146 and sends the same to the converter 142 and the inverter 143.

The fourth power supply unit 140 further comprises a first condenser 148a which reduces ripple of the rectified power received from the rectifier 141 and then supplies its power to the converter 142; a second condenser 148b which reduces ripple of the stepped-up DC power and then supplies its power to the inverter 143.

The input AC power is converted by the rectifier 141 into a DC power, which, in turn, is stepped up by the converter 142, and then the inverter 143 modulates the stepped up DC power into an AC power of a pulsed electromagnetic field, which, in turn, is stepped up by the pulse transformer 145 to supply the stepped up power to the reverse polarity converter 200.

And inside the control unit 146 is provided a separate input interface (not shown), with which a user can provide such information as a current value, a voltage value, frequency of the power to be supplied to the first and second quantum energy generating coils 360a, 360b, power supply time and downtime (timer function), switching function, a surface temperature of the quantum energy generating coils 360a, 360b as well as of a user's body part where the device is applied. For the timer function, a user can select, by using the input interface, any one of the sections for power supply period, for example, sections of 30 seconds to 3 minutes, 30 seconds to 10 minutes, 30 seconds to 20 minutes, 30 seconds to 30 minutes, and any one of the sections for stop time, for example, sections of 30 seconds to 3 minutes, 30 seconds to 10 minutes, 30 seconds to 20 minutes, and 30 seconds to 30 minutes.

And the quantum energy generating coils 360a, 360b, as integrated with a user's garment (a hat 410, a top 420, a pair of glove 430, a bottom 440, a pair of socks 450, or a pair of shoes (not shown)) transmit, in real time, the information of a temperature of their own as well as of the user's body part (the head, torso, hands, lower body, and feet) where the device is applied, as detected by a plurality of temperature sensors (not shown), via a conductive wire (not shown), to the control unit 146, which, in turn, analyzes the data of the temperature detected and, controls the output power of the power supply unit 140 supplying power to the first and second quantum energy generating coils 360a, 360b so as to maintain the temperature at a set level.

With such information as power supply time and downtime input in a way as described above, the device can be used for a long time even with a limited capacity of the battery charged a single time and irradiate the quantum energy to a human body in a form of pulsed waves.

The fourth power supply unit 140, as shown in FIG. 11a, is stored in a separate pocket (not shown) made and located, inside or outside, at one side of a lower front part or one side of a lower rear part of a top covering a torso (the thorax and belly, etc.).

FIG. 3d is a sectional view of the fifth power supply unit as shown in FIG. 1.

With reference to FIG. 3b, the fifth power supply unit 150 comprises: a power supply 151 of either of an AC power supply 151a and a DC power supply 151b, an AC/DC converter 152, an AST (Automatic Transfer Switch) 153, a low frequency outputting unit 154, a switching device 155, a control unit 156 with such control capabilities as a temperature sensor (not shown) detecting surface temperature of quantum energy generating coils 360a, 360b as well as surface temperature of the body part, a pulse width modulation function, a pulse density modulation function, and a pulse repetition rate control function.

The AC power supply unit 151a of the power supply unit 151 supplies an AC power of 220 volts at 6 Hz to the step down transformer (not shown), which, in turn, supplies a stepped down power, via an AST (Automatic Transfer Switch) 153, to the low frequency generating and outputting unit 154 with an oscillator 154a, an frequency allocation unit 154b, a control unit 154c, an amplifying unit 154d integrated, or the DC power supply unit 151b (the battery) supplies a DC power of a range from 6 to 50 volts, via the ATC 153, to a low frequency generating and outputting unit 154 with an oscillator 154a, a frequency allocation unit 154b, a control unit 154c, an amplifying unit 154d integrated, wherein the oscillator 154a, according to the data programmed and input to the control unit 156, in advance, as required for treatment, generates an appropriate electromagnetic wave to be applied to the first and second quantum energy generating coils 360a, 360b and supplies the same to the frequency allocation unit 154b, which, in turn, converts the electromagnetic wave generated by the oscillator 154a into a low frequency signal such that the first and second quantum energy generating coils 360a, 360b could generate electromagnetic fields in opposing directions to each other so as to make the two fields overlapping thereby generating enough quantum energy in a state of a zero strength of the fields, and supplies the same to the control unit 154c, which, in turn, adjusts the pulse of the low frequency signal generated by the frequency allocation unit 154c or the strength of a low frequency or a high frequency, based on controls of the control unit 156, and then applies the same to the amplifying unit 154d, which, in turn, amplifies the low frequency pulse signal received from the control unit 154c and supplies the amplified power of a form of a pulsed electromagnetic field, via the switching device 155, to the reverse polarity converter 200 such that the first and second quantum energy generating coils 360a, 360b could generate quantum energy enough for an effective treatment.

And to the control circuit of the control unit 156 is further provided such functions as power supply time and downtime (timer function), switching devices, etc.

The battery is one type selected from a group of types of rechargeable (secondary) batteries consisting of a nickel-cadmium (Ni—Cd) battery, an alkaline battery, a nickel metal hybrid battery (Ni-MH), a sealed lead-acid battery (SLA), a lithium-ion battery, and a lithium polymer battery.

The fifth power supply unit 150, as shown in FIG. 11a, is stored in a separate pocket (not shown) made and located, inside or outside, at one side of a lower front part or one side of a lower rear part of a top covering a torso (the thorax and belly, etc.).

The data to be supplied to the input interface of the first, second, third, fourth, and fifth power supply units 110, 120, 130, 140, 150 as well as to the control unit are determined in such a way that with a quantum resonance spectrometer an electromagnetic wave corresponding to that of an organ selected as a target of analysis is applied to the body of a user of the present disclosure to detect and analyze a weak electromagnetic energy, as resonating with the electromagnetic wave, of the user so as to determine whether the bio-data of the user thus obtained indicates any abnormalities, more particularly, determines an amount of quantum energy to be irradiated enough to recover or treat an abnormal state based on a comparison of a resonance value of an organ of the body under a predetermined level with a reference value of electromagnetic resonance or electromagnetic energy for a healthy state.

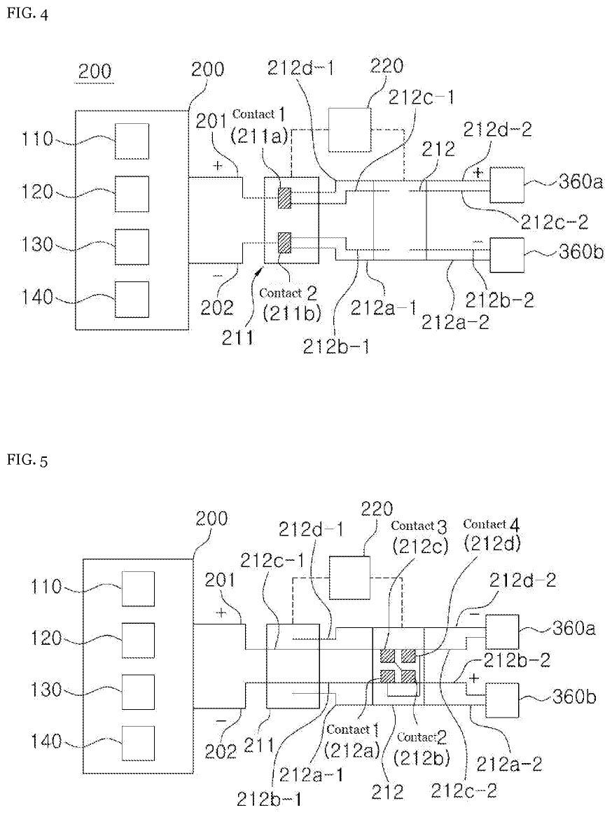

FIG. 4 is a schematic diagram illustrating a straight polarity of a reverse polarity converter as shown FIG. 1, and FIG. 5 is a schematic diagram illustrating a reverse polarity of a reverse polarity converter as shown FIG. 1.

With reference to FIGS. 4 and 5, the reverse polarity converter 200 comprises: a first switch having a contact 1 211a and a contact 2 211b, connected to, and controlled by, a micro controller 220; a second switch having a contact 1 212a, a contact 2 212b, a contact 3 212c, and a contact 4 212d, connected to, and controlled by, a micro controller 220; input wires 201, 202 of the first switch 212 and the second switch 211; input wires 212a-1, 212b-1, 212c-1, 212d-1 and output wires 212a-2, 212b-2, 212c-2, 212d-2 of the second switch 212.

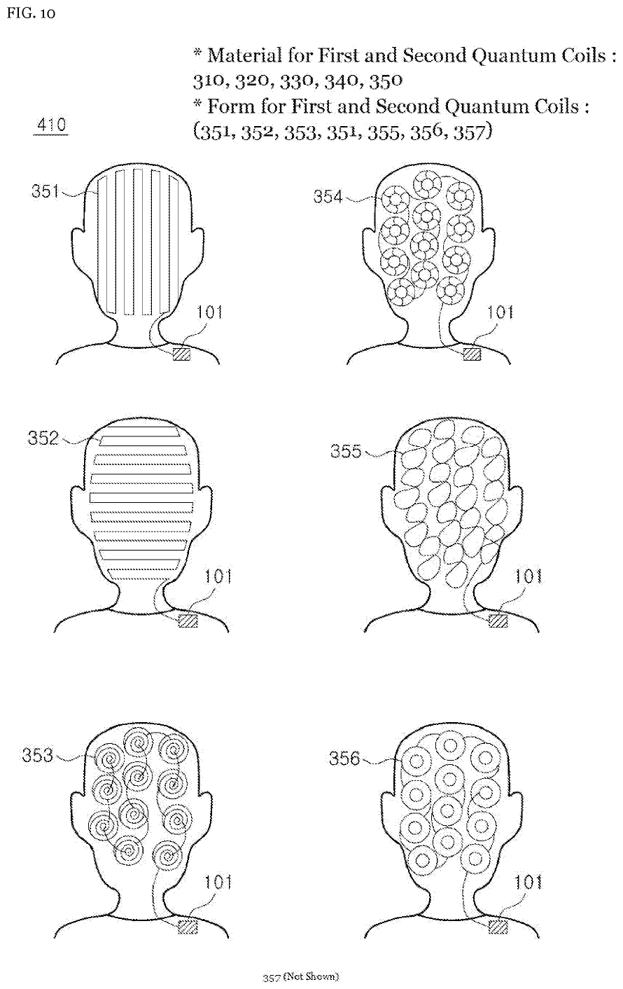

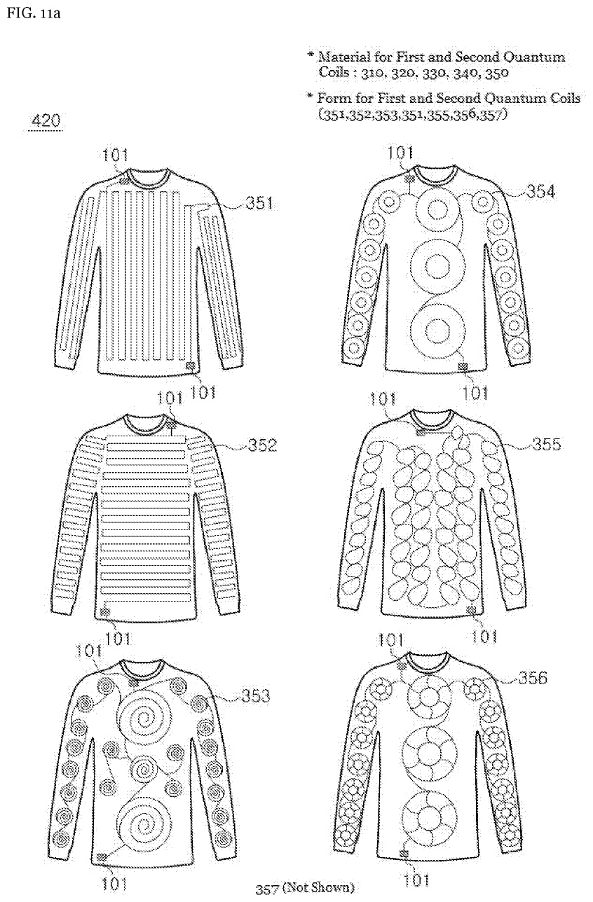

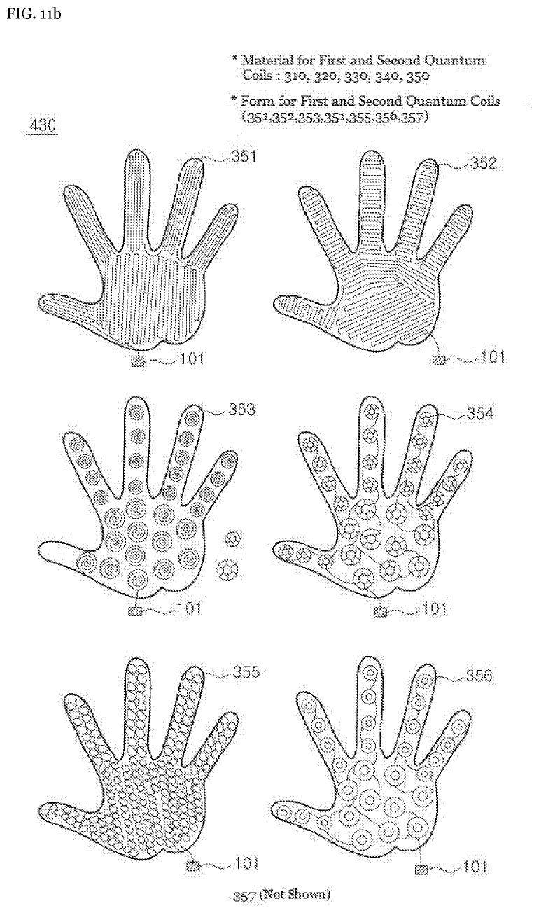

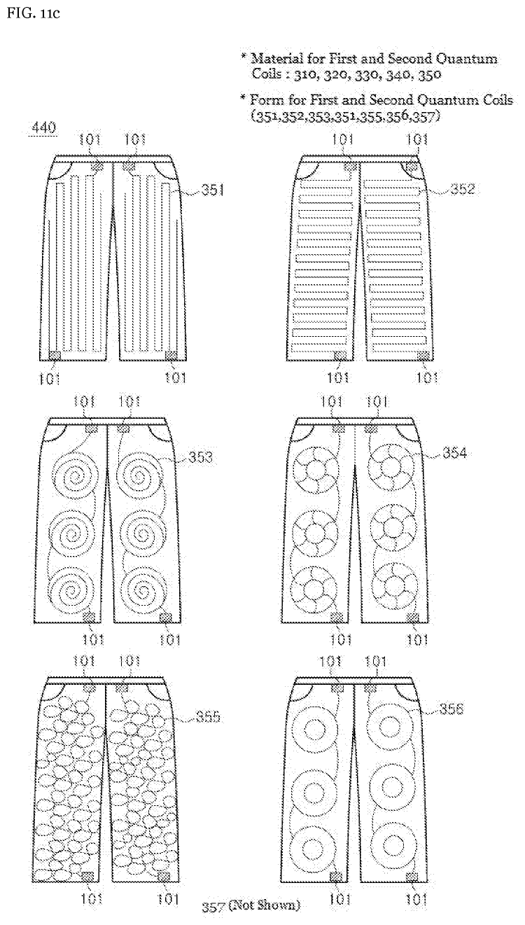

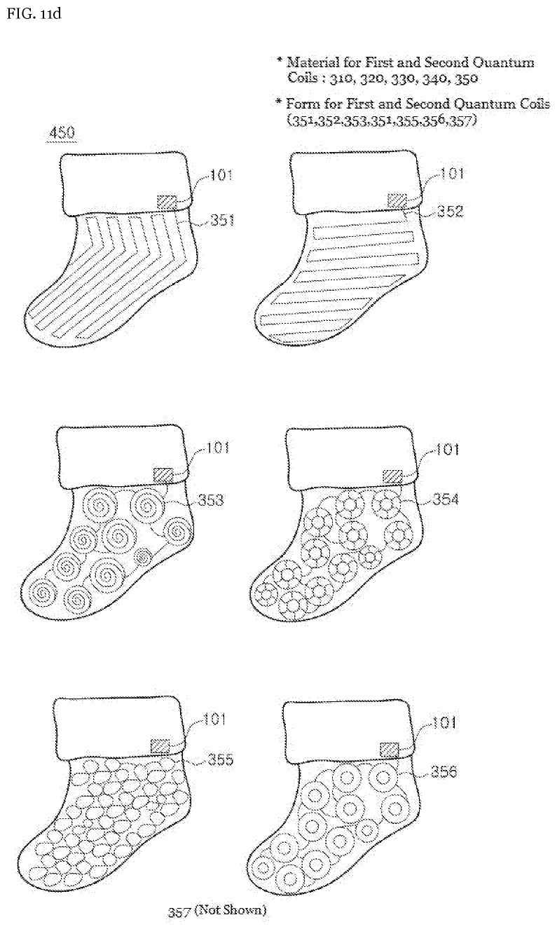

With reference to FIG. 4, any one power supply unit of the power supply 200, as selected from a group of the first 110, second 120, third 130, fourth 140, and fifth 150 power supply units, supplies a variable (current or voltage) DC power, via a positive output wire 201 and a negative output wire 202, to the first switch 211, as controlled by the micro controller 220, which, in turn, supplies, without change of polarity (that is, as a straight polarity), via the input wires 212a-1, 212d-1 and the output wires 212a-2, 212d-2 of the second switch, a variable power to a contact 101 of the first and second quantum energy generating coils 360a, 360b integrated with a hat as shown in FIG. 10; a contact 101 of the same coils integrated with a top as shown in FIG. 11a; a contact 101 of the same coils integrated with a pair of gloves as shown in FIG. 11b; a contact 101 of the same coils integrated with a bottom as shown in FIG. 11c; or a contact 101 of the same coils integrated with a pair of socks or shoes as shown in FIG. 11d, such that the variable power is supplied, via a conductive wire (not shown) and contacts 101, to the first and second quantum energy generating coils 360a, 360b disposed in one or more, or all, of the areas including the hat covering the head, the top covering the torso, the pair of gloves for hands, the bottom covering from the hip joint to the legs, the pair of socks for feet, in such a way that variable electromagnetic fields are created, in directions perpendicular to that of the current, by the variable power supply unit, and irradiated to the user's body while the first and second quantum energy generating coils 360a, 360b, as they are disposed in opposing directions to each other, generates variable electromagnetic fields in opposing directions, which overlap each other and thus dissipate to create and irradiate quantum energy in a state of zero strength electromagnetic fields onto the center of the user's body.

With reference to FIG. 5, a variable (current or voltage) DC power from a power supply unit 200 selected from a group of power supply units consisting of a first power supply unit 100, a second power supply unit 120, a third power supply unit 130, a fourth power supply unit 140, a fifth power supply unit 150, is provided first, via a positive conductive wire 201 and a negative conductive wire 202, to a first uncontrollable switch 211 and input wires 212a-1, 212d-1 of a second switch 212 controlled by a micro controller, by control of which a contact 3 212c of the second switch comes into contact with a contact 2 212b, and a contact 1 212a comes into contact with a contact 4 212d, thereby reversing the polarities (that is, a negative becomes a positive, and vice versa), whereby a variable power is applied to the first and second quantum energy generating coils 360a, 360b via output wires 212b-2, 212d-2 of the second switch.

The polarity reversing period of the first switch 211 and the second switch 212, as selected by a user from ranges of 1 to 10 seconds; 10 to 30 seconds; 30 seconds to 5 minutes; 5 to 20 minutes, etc., may be provided to the control circuit of the micro controller, based on which a variable power is supplied to a contact 101 of the first and second quantum energy generating coils 360a, 360b integrated with a hat as shown in FIG. 10; a contact 101 of the same coils integrated with a top as shown in FIG. 11a; a contact 101 of the same coils integrated with a pair of gloves as shown in FIG. 11b; a contact 101 of the same coils integrated with a bottom as shown in FIG. 11c; or a contact 101 of the same coils integrated with a pair of socks or shoes as shown in FIG. 11d, such that the variable power is supplied, via a conductive wire (not shown) and contacts 101, to the first and second quantum energy generating coils 360a, 360b disposed in one or more, or all, of the areas including the hat covering the head, the top covering the torso, the pair of gloves for hands, the bottom covering from the hip joint to the legs, the pair of socks for feet, in such a way that variable electromagnetic fields are created, in directions perpendicular to that of the current, by the variable power supply unit, and irradiated to the user's body while the first and second quantum energy generating coils 360a, 360b, as they are disposed in opposing directions to each other, generates variable electromagnetic fields in opposing directions, which overlap each other and thus dissipate to create and irradiate quantum energy in a state of zero strength electromagnetic fields onto the center of the user's body.

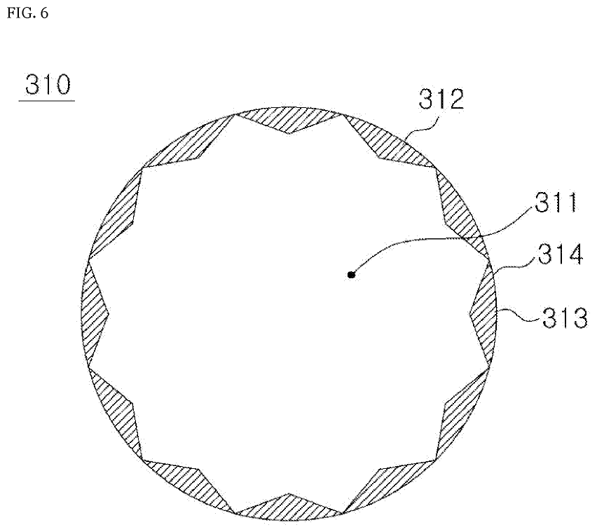

FIG. 6 is a sectional view of a first material configuration of the first and second quantum energy generating coils of the wearable quantum treatment device as shown in FIG. 1.

With reference to FIG. 6, the material of the first and second quantum energy generating coils 360a, 360b must be a conductive, optionally heat emitting, material; any material if conductive and optionally heat emitting may be used for the purpose. The first material having conductive properties may be a conductive fabric, which comprises a porous fabric structure 311; pores 312 therein; a conductive material layer 313 to be filled into the pores; a resin layer 314 covering the conductive material layer.

The fabric of the porous fabric structure may be made of any one selected from a group of materials consisting of nylon, polyamide, polyaramid, polyetherimide, polyacrylonitrile, polyaniline, polyethyleneoxide, polyethylene naphthalate, polybutylene terephthalate, styrene butadiene rubber, polystyrene, polyvinyl chloride, polyvinyl alcohol, polyvinylidene, fluoride, polyvinyl butylene, polyurethane, polybenzoxazole, polybenzimidazole, polyamide imide, polyethyleneterephthalate, polyethylene, polypropylene, polycarbonate, copolymers and mixtures thereof.

The pores 312 of the porous fabric structure is formed directly in the porous fabric structure.

The conductive material may be selected from a group consisting of such metals as iron (Fe), zirconium (Zr), beryllium (Be), manganese (Mn), germanium (Ge), zinc (Zn), palladium (Pd), copper (Cu), rhodium (Rh), magnesium (Mg), tin (Sn), silver (Au), bismuth (Bi), aluminium (Al), lithium (Li), iridium (Ir), ruthenium (Ru), platinum (Pt), titanium (Ti), osmium (Os), gallium (Ga), rubidium (Rb), indium (In), and metal nano-wires, conductive polymers, with a metal content being 30 to 70% relative to a total amount of the conductive material, and, for the purpose of imparting heat emitting properties, any one selected from the group consisting of carbon nanotube (CNT), graphene, carbon black is added and mixed in an amount of 30 to 70% relative to a total amount of the conductive material.

The resin layer 314 is made of any one selected from a group consisting of silicon oxide, silicon nitrile, aluminium oxide, acrylic resin, epoxy resin, silicon resin, fluoropolymer, styrene resin, polyolefin resin, thermoplastic elastomer, polyoxy alkylene resin, polyester resin, polyvinyl chloride resin, polycarbonate resin, polyphenylene sulfide resin, polyamide resin, polysilicon epoxy, and mixtures thereof.

One or more selected from the group consisting of oxane solvent, amine solvent, polyol solvent, alkane solvent, benzene solvent, ether solvent are, in an appropriate amount, added to a reactor with a mixer; and one or more selected from the group of conductive materials consisting of carbon black (C), acetylene black (C), Ketjenblack (C) are in an appropriate amount (30 to 90%) added thereto and, while being mixed by a mixer (not shown), an appropriate amount of metals such as zinc (Zn), paladium (Pd), copper (Cu), Rhodium (Rh), magnesium (Mg), etc. is added thereto; one or more of the group of materials, for the resin layer 304, consisting of silicon oxide, silicon nitrile, aluminium oxide, etc are in an appropriate amount added and mixed by the mixer, and then one or more selected from the group of anti-corrosive agents consisting of benzotriazole, butylbenzyltriazole, 2-aminopyrimidine or 5,6-dimethyl benzimidazole, or 2-amino-5-mercapto-1,3,4-thiadiazole, 2-mercapto pyridine, 2-mercaptobenzoxazole, 2-mercapto phenol, alkyl dithiothiadiazole, 4-methyl phthalate, phenyl mercapto-tetrazole, tetrachlorophthalate are added, in an appropriate amount, and mixed by the mixer, and then perfluoroalkyl carboxylate is added to adjust liquidity of the mixture, and then such acrylic resins as polyacrylate or polyacrylate ester and such cellulosic resin as ethyl cellulose, such binder resin as polyurethane resin are added as a dispersing agent, in an appropriate amount, and mixed by the mixer, and further such surfactants as sodium lauryl sulfate and such wetting and dispersing agents as polyethylen glycol are added, in an appropriate amount, and continuously mixed by the mixer, and, after adjusting viscosity of the composition for a conductive material layer, the porous fabric structure made of one or more selected from the group consisting of nylon, polyimide, polyarimid, polyetherimide, polyacrylonitrle, polyaniline, polyethyleneoxide, polyethylene naphthalate, polybutylene terephthalate, styrene butadiene rubber, polystyrene, polyvinyl chloride, polyvinyl alcohol, polyvinylidene, fluoride, polyvinyl butylene, polyurethane, polybnezoxazole, polybenzimidazole, polyamide imide, polyethyleneterephthalate, polyethylene, polypropylene, polycarbonate, copolymers and mixtures thereof is processed by one selected from the group consisting of impregnation, screen printing, spray coating, doctor blade coating, gravure coating, roll to roll (R2R) coating, so as to form a conductive material layer, and then heat treated in an oven (not shown) at a temperature of 80 to 150° C. The above method is one example to form a conductive material on the porous fabric; the present disclosure should not be construed as being limited to the above method, but as being inclusive of other known methods.

The first and second quantum energy generating coils 360a, 360b may be formed in a state that the porous fabric structure has been extended so as to prevent sagging thereof. In case the conductive material layer is formed in a vertical direction, the porous fabric structure may be extended in a vertical direction and then the conductive material layer is formed. When extending the porous fabric structure, the entire edges may be fixed so as to provide tensile force to the porous fabric structure. Considering its physical properties, the porous fabric structure should be extended not more than 1.3 times the original length to prevent too much extension.

After the conductive material layer has been formed, the porous fabric structure may be restored to its original state so as to form a denser arrangement of the coils for the first and second quantum energy generating coils 360a, 360b, to guarantee a continuous flow of current even if the porous fabric structure is extended, and eventually to securely generate electromagnetic fields in directions perpendicular to the direction of the current, such that the quantum energy generating coils wound in opposing directions generate electromagnetic fields in opposing directions thereby two fields overlapping and dissipating to create and irradiate quantum energy to important acupuncture points and the body of a user in a state of zero strength electromagnetic fields.

The windings of the first and second quantum energy generating coils 360a, 360b are made in directions opposing to each other, and the windings may be made in one or more forms selected from the group of vertical or lateral zigzag forms, a transformed solenoid coil form, a toroid coil form, a Möbius coil form, and a Helmholtz coil form, and then disposed on the porous fabric structure made of fabric or non-woven fabric.

The first and second quantum energy generating coils 360a, 360b may be wound such that the coils are formed with moderate curves in such areas as less susceptible to external stresses, but with denser patterns in such areas more susceptible to external stresses as joints such as elbows and knees or those parts which are often folded or bear shocks.

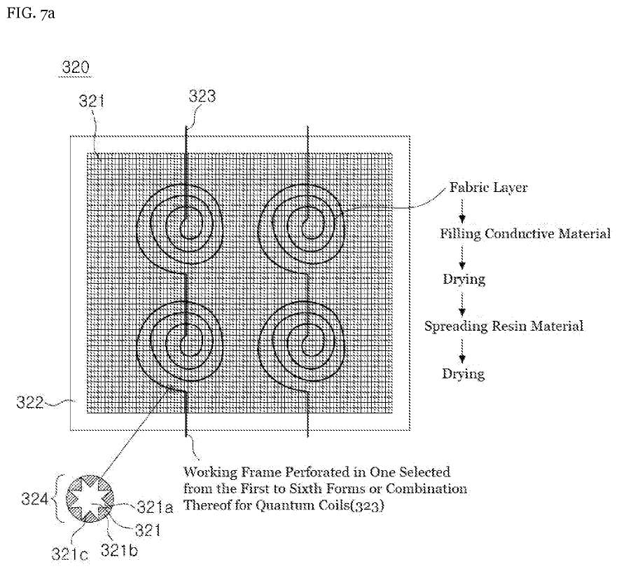

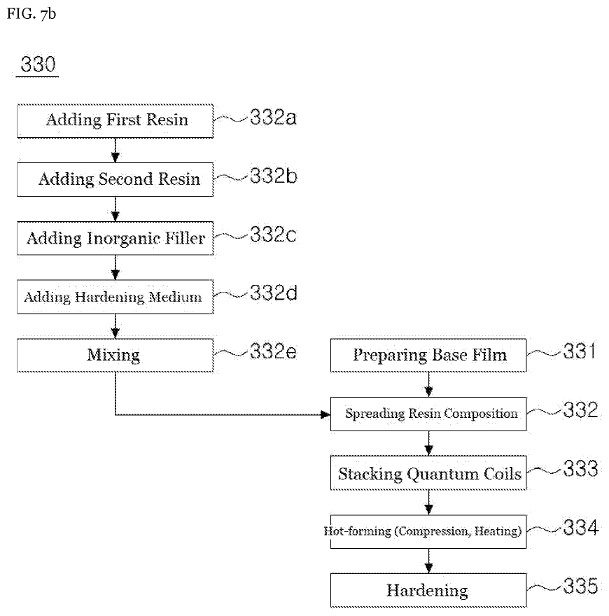

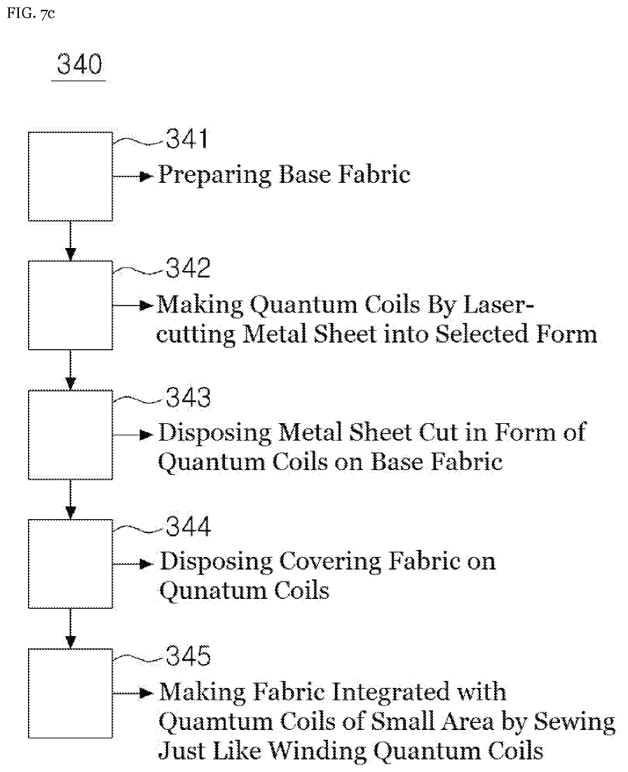

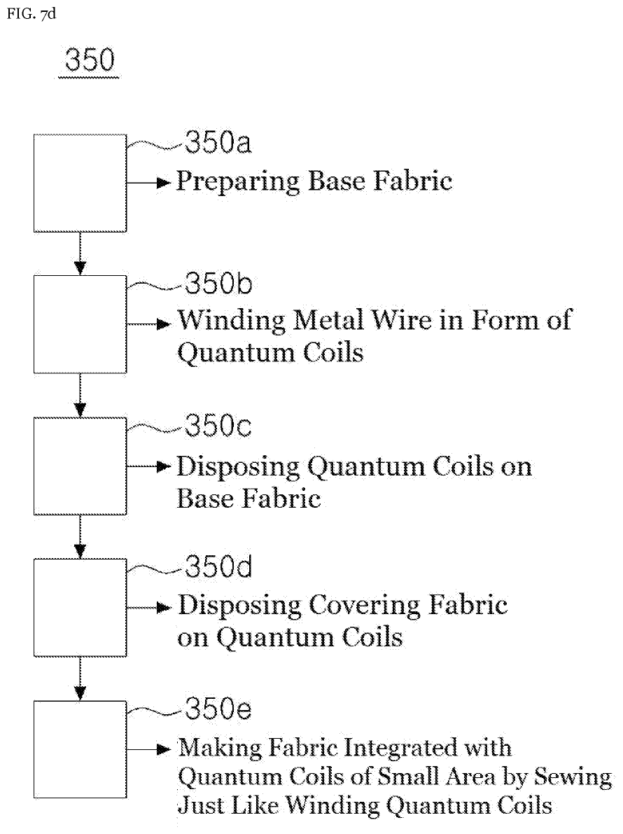

FIG. 7a is a sectional view of a second material configuration of the first and second quantum energy generating coils of the wearable quantum treatment device as shown in FIG. 1.

With reference to FIG. 7a, the material of the first and second quantum energy generating coils 360a, 360b must be a conductive, optionally heat emitting, material; any material if conductive and optionally heat emitting may be used for the purpose.

The first and second quantum energy generating coils 360a, 360b may be configured and made by a method, wherein a fabric 321 for the porous fabric structure is spread on a working table 322, and then a frame 323 perforated in a form of the first and second quantum energy generating coils 360a, 360b as shown in FIG. 5 selected from the sixth form as shown in FIG. 9e, and a seventh form 357 as a combination of the first to sixth forms is disposed in contact with the fabric prepared, and the fabric 321a of the porous fabric structure, as made with a process as shown in FIG. 6, is made of one material selected from the group consisting of nylon, polyimide, polyaramid, polyetherimide, polyacrylonitrile, polyaniline, polyethyleneoxide, polyethylene naphthalate, polybutylene terephthalate, styrene butadiene rubber, polystyrene, polyvinyl chloride, polyvinyl alcohol, polyvinylidene, fluoride, polyvinyl butylene, polyurethane, polybenzoxazole, polybenzimidazole, polyamide imide, polyethyleneterephthalate, polyethylene, polypropylene, polycarbonate, copolymers and mixtures thereof; the material to be filled into the pores 321b of the porous fabric structure is made of one or more selected from the group consisting of such metals as iron (Fe), zirconium (Zr), beryllium (Be), manganese (Mn), germanium (Ge), zinc (Zn), palladium (Pd), copper (Cu), rhodium (Rh), magnesium (Mg), tin (Sn), silver (Au), bismuth (Bi), aluminium (Al), lithium (Li), iridium (Ir), ruthenium (Ru), platinum (Pt), titanium (Ti), osmium (Os), gallium (Ga), rubidium (Rb), indium (In), and metal nano-wires, conductive polymers, with a metal content being 30 to 70% relative to a total amount of the conductive material, and, for the purpose of imparting heat emitting properties, any one selected from the group consisting of carbon nanotube (CNT), graphene, carbon black is added and mixed in an amount of 30 to 70% relative to a total amount of the conductive material; and then the selected conductive metals and the materials for imparting heat emitting properties are put into a reactor (not shown) with a mixer to be mixed for a predetermined time to adjust the viscosity of the mixture, which is sprayed, by using a sprayer (not shown) partially onto a form selected from the first to sixth forms for the first and second quantum energy generating coils 360a, 360b, and a seventh form (not shown) as a combination of the first to sixth forms, and then dried by using a dryer (not shown) for 1 to 2 hours at a temperature of 80 to 120° C., and then the fabric 321, as produced in such a way as described above and cooled down, is spread on the working table 322, and then a frame 323, as perforated in a form selected from the group of the first to sixth form for the first and second quantum energy generating coils 360a, 360b, and a seventh form as a combination of the first and sixth forms, is disposed in contact with the fabric prepared, and then into a reactor with a mixer is put a material, for forming a resin layer 321c, selected from the group consisting of silicon oxide, silicon nitrile, aluminium oxide, acrylic resin, epoxy resin, silicon resin, fluoropolymer, styrene resin, polyolefin resin, thermoplastic elastomer, polyoxy alkylene resin, polyester resin, polyvinyl chloride resin, polycarbonate resin, polyphenylene sulfide resin, polyamide resin, polysilicon epoxy, and mixtures thereof, and then, while being mixed in the reactor with a mixer, a solvent selected from the group consisting of oxane solvent, amine solvent, polyol solvent, alkane solvent, benzene solvent, ether solvent are, in an appropriate amount, added to a reactor with a mixer and mixed to adjust the viscosity of the mixture, which, in turn, is sprayed partially onto a form selected from the group consisting of the first 351 and sixth 356 forms for the first and second quantum energy generating coils 360a, 360b, and a seventh form 357 (not shown) so as to form a resin layer 321C for coating, which then is dried and cooled down by a dryer (not shown) for 1 to 2 hours at a temperature of 80 to 120° C., thereby eventually producing a fabric 324 with conductive and optionally heat emitting properties.

The above method is one example to form a conductive material on the porous fabric; the present disclosure should not be construed as being limited to the above method, but as being inclusive of other known methods.

FIG. 7b is a sectional view of a third material configuration of the first and second quantum energy generating coils of the wearable quantum treatment device as shown in FIG. 1.

With reference to FIG. 7b, the material of the first and second quantum energy generating coils 360a, 360b must be a conductive, optionally heat emitting, material; any material if conductive and optionally heat emitting may be used for the purpose.

A third method for making the material for the first and second quantum energy generating coils 360a, 360b is a method of using a flexible printed circuit board.

The method for making the material for the first and second quantum energy generating coils 360a, 360b with a flexible printed circuit board comprises steps of: preparing a base film 331; spreading a resin composition 332; stacking the coils 333; hot-forming 334; hardening 335.

The material of the base film prepared by a step of preparing a base film 331 is selected from the group consisting of polyimide, PET (polyethylene terephthalate), PMMA (polymethyl methacrylate), PDMS (polymethylsiloxane), polyester film, polyethylene film, polypropylene film.

The base film, so as to be suitable for foldable parts of a body, is made in one selected from a group consisting of a square, a rectangle, a circle, a polygonal, or any other shapes, and the area thereof may be variously determined to be such as 1 cm2, 4 cm2, 9 cm2, 16 cm2, 25 cm2, 36 cm2.

The step 332 of spreading a resin composition may be divided into sub-steps of: adding a first resin 332a; adding a second resin 332b; adding an inorganic filler 332c; adding a hardening medium 332d; stirring 332e.

The first resin material, as added at the step 332a of adding a resin, may be selected from the group consisting of polyimide, PET (polyethylene terephthalate), PMMA (polymethyl methacrylate), PDMS (polymethylsiloxane), polyester film, polyethylene film, polypropylene film; The second resin material, as added at the step 332a of adding a second resin, may be selected from the group consisting of such poly-functional acid anhydrides as tetrahydrophthalic anhydride, hexahydrophthalic anhydride, methyltetrahydrophthalic anhydride, PDMA(pylomellitic dianhydride), BTDA(benzophenon teyracarboxylic dianhydride), TMEG(ethylene glycol bis-trimellitic dianhydride), TMTA(glycerol tris-trimellitic dianhydride), MCTC(5-(2,5-dioxotetrahydropryle)-3-cyclohexene-1,2-dicarboxylic anhydride), TDA(2,5-dioxotetranhydrofuran-3-yl)1,2,3,4-tetrahydronaphthalene-1,2-dicarboxyl anhydride), and then is added into the reactor (not shown); and the inorganic filler material, as added at the step 332c, may be selected from a group consisting of such conductive materials as carbon black (C), acetylene black (C), Ketjenblack (C), and added in an appropriate amount (30 to 90%); any one of the group of nitride filler, alumina, silica as well as a hardening agent is added in an amount of 30 to 70% relative to a total amount of inorganic materials added and then mixed by a mixer 332e for a predetermined time so as to achieve a predetermined level of viscosity; and then the resin composition prepared as above is sprayed, with a predetermined thickness, by using a high pressure spray gun onto a base film of a predetermined area, as prepared at the step 321 of preparing a base film, made of one selected from the group consisting of polyimide, PET (polyethylene terephthalate), PMMA (polymethyl methacrylate), PDMS (polymethylsiloxane), polyester film, polyethylene film, polypropylene film.

In the step of stacking the quantum energy generating coils, the first and second quantum energy generating coils 360a, 360b are made by a process comprising: selecting any one material selected from the group consisting of copper (Cu), silver (Ag), gold (Au), etc.; machining, by laser-cutting, etching or plating, the selected material in any one of the group consisting of a first, second, third, fourth, fifth, sixth forms and a seventh form as a combination thereof 357; disposing the machined material in contact with a base film with the resin composition spread thereon with a predetermined thickness, which is then stacked on a lower mold (not shown); compressing the stack by lowering an upper mold (not shown), thereby producing the first and second quantum energy generating coils 360a, 360b.

FIG. 7c is a sectional view of the fourth material configuration of the first and second quantum energy generating coils of the wearable quantum treatment device as shown in FIG. 1.

With reference to FIG. 7c, the material of the first and second quantum energy generating coils 360a, 360b must be conductive and optionally heat emitting, so any material having conductive and optionally heat emitting properties may be used; the fourth material configuration of the first and second quantum energy generating coils 360a, 360b is made by a process comprising: first preparing a porous base fabric 341; selecting any one material from the group consisting of copper (Cu), stainless steel (STS304, STS316L), silver (Ag), titanium (Ti), etc. as in a form of disc or carbon sheet molded with a high compression; machining 342, by laser-cutting, the selected material into any one form selected from the group consisting of first 351 to sixth 356 forms and a seventh form 357 as a combination thereof for the quantum energy generating coils; disposing the machined coils on, and in contact with, the porous base fabric 341; stacking a covering fabric on the quantum energy generating coils 360a, 360b; sewing up 345, by a sewing machine (not shown) the stack just like winding the coils, thereby eventually producing a fabric of a predetermined area having conductive and optionally heat emitting properties.

FIG. 7d is a sectional view of the fifth material configuration of the first and second quantum energy generating coils of the wearable quantum treatment device as shown in FIG. 1.

With reference to FIG. 7d, the material of the first and second quantum energy generating coils 360a and 360b must be conductive and optionally heat emitting, so any materials having conductive and optionally heat emitting properties may be used; the fifth material configuration of the first and second quantum energy generating coils 360a, 360b is made by a process comprising: preparing a base fabric 350a; selecting any one from the group, of materials conductive and heat emitting with a diameter of 50 um to 2 mm, consisting of silver, copper, nickel, carbon fiber, graphite fiber, medium temperature carbonized fiber, activated carbon fiber; winding 350b the material covered with an insulating material or a sheathed heat emitting material (carbon fiber, nichrome wire) in any one selected from the group consisting of the first to sixth forms and a seventh form as a combination thereof for the first and second quantum energy generating coils 360a, 360b on, and in contact with, the base fabric 350a; stacking 350d a covering fabric thereon; sewing, by using a sewing machine (not shown), the stack to fix the quantum energy generating coils; cutting, by using a cutter (not shown), the stack into a plurality of quantum energy generating coils of a predetermined area.

The first material 310 of the quantum energy generating coils 360a, 360b as shown in FIG. 6, the second material thereof as shown in FIG. 7a, the third material thereof as shown in FIG. 7b, the fourth material thereof as shown in FIG. 7c, the fifth material thereof as shown in FIG. 7d have heat emitting properties, which can increase the body temperature of the user of the present device, thereby improving activity of body cells, the immune system, blood cells, and blood circulation, when the body temperature increases over a normal level (36.5° C.), reaching its peak level at 42° C.

Conversely, when the temperature of the human body decreases below a normal level, the activity of body cells, immune system, and blood cells and blood circulation also decrease, which causes a variety of diseases as confirmed by many research results in the field. Unlike the thermal characteristics of human body cells, the immune system and blood cells, the harmful bacteria causing diseases to the body become less active when the body temperature increases over a normal level (36.5° C.); more particularly, influenza is annihilated at 39.5° C., cancer cells, pathogens of skin diseases, tuberculosis bacteria die at 44° C. And any one power supply unit of the power supply 200 selected from the first 110, second 120, third 130, fourth 140, fifth 150 power supply units supplies a variable power to the quantum energy generating coils 360a, 360b, which, in turn, generate electromagnetic fields in directions perpendicular to the direction of the current, wherein the heat emitting of the quantum energy generating coils 360a, 360b becomes more active as thermal energy is applied to the electromagnetic fields; a plurality of the quantum energy generating coils 360a, 360b disposed at the front and rear or at both sides of the body of a user are wound in directions opposing each other, whereby the electromagnetic fields are generated in directions opposing each other, thereby overlapping and dissipating other other to create and boost quantum energy for treatment in a state of a zero strength electromagnetic field around the center of the body of a user.

FIG. 8 is a sectional view of a first form of the first and second quantum energy generating coils of the wearable quantum treatment device as shown in FIG. 1.

With reference to FIG. 8, the first form 351 of the first and second quantum energy generating coils 360a, 360b is a form wherein a coil is wound from top to bottom, with intervals, forming a zigzag pattern; the overall pattern has fine creases to prevent damages to the coils from distortions caused by the extensions or bending of the body.

FIG. 9a is a sectional view of a second form of the first and second quantum energy generating coils of the wearable quantum treatment device as shown in FIG. 1.

With reference to FIG. 9a, the second form 352 of the first and second quantum energy generating coils 350a, 350b is a form wherein a coil is wound, laterally, with intervals, forming a zigzag pattern; the overall pattern has fine creases to prevent damages to the coils from distortions caused by the extensions or bending of the body.

FIG. 9b is a sectional view of a third form of the first and second quantum energy generating coils of the wearable quantum treatment device as shown in FIG. 1.

With reference to FIG. 9b, the third form 353 of the first and second quantum energy generating coils 350a, 350b is a form wherein a solenoid coil is transformed into a shape of disc; the overall pattern has fine creases to prevent damages to the coils from distortions caused by the extensions or bending of the body with concentric circles of coils wound from the center outwardly or from the circumference inwardly.

FIG. 9c is a sectional view of a fourth form of the first and second quantum energy generating coils of the wearable quantum treatment device as shown in FIG. 1.

With reference to FIG. 9c, the fourth form 354 of the first and second quantum energy generating coils is a form wherein a coil is wound in a toroidal shape; the overall pattern has fine creases to prevent damages to the coils from distortions caused by the extensions or bending of the body with the coils wound in a shape of helix with winding spacings.

FIG. 9d is a sectional view of a fifth form of the first and second quantum energy generating coils of the wearable quantum treatment device as shown in FIG. 1.

With reference to FIG. 9d, the fifth form 355 of the first and second quantum energy generating coils 360a, 360b is a form wherein a coil is wound in a Möbius shape; the overall pattern has fine creases to prevent damages to the coils from distortions caused by the extensions or bending of the body in a shape of double helix like a number “8”.

FIG. 9e is a sectional view of the sixth form of the first and second quantum energy generating coils of the wearable quantum treatment device as shown in FIG. 1.

With reference to FIG. 9e, the sixth form 356 of the first and second quantum energy generating coils 350a, 350b is a form wherein a coil is wound in a shape of Helmholtz coil; the overall pattern has fine creases to prevent damages to the coils from distortions caused by the extensions or bending of the body with a shape of double circle.

FIG. 10 is a sectional view of the head 410 of a user wearing a head cover or a cap integrated with first and second quantum energy generating coils 360a, 360b, as embodied in the first 351, second 352, third 353, fourth 354, fifth 355, sixth 356 forms and a seventh form 357 as a combination of the first to sixth forms as shown in FIGS. 8, 9a, 9b, 9c, 9d, and 9e, of the wearable quantum treatment device.

With reference to the drawings enclosed, the first and second quantum energy generating coils 360a, 360b integrated with a cover or hat for the head may be made in any one selected from the group consisting of: weaving a garment with a conductive fabric, partially spraying a conductive material, through a perforated frame having a form of a quantum energy generating coil, onto a normal fabric; attaching the device to a fabric of a flexible printed circuit board having a form of a quantum energy generating coil; laser cutting a conductive metal sheet into a form of a quantum energy generating coil to insert or stack the same between an upper fabric and a lower fabric and then sewing them together; or winding a conductive metal wire or a sheathed electric wire over a base fabric to make a quantum energy generating coil, which is then stacked and sewing with a covering fabric.

And when the quantum energy generating coils 360a and 360b are integrated with a cover or a hat for the head 410, a plurality of temperature sensors (not shown) for detecting a temperature of the body part (head) where the device is applied as well as the quantum energy generating coils 360a, 360b are also provided with intervals therebetween; the temperatures detected as above are provided, in real time, via a conductive wire (not shown), to a control unit of any one selected from the group consisting of: the first 110, second 120, third 130, fourth 140, fifth 150 power supply units, which, in turn, controls the output power of the selected power supply unit.

And the plurality of first and second quantum energy generating coils 360a, 360b integrated with a cover or a hat for the head are disposed in such areas of the head of a user as the forehead and the back or both sides of the head so as to make the two groups of the coils facing each other, and the windings of the two groups of coils are made in directions opposing each other so that power from any one of the power supply 100 selected from the group consisting of the first 110, second 120, third 130, fourth 140, fifth 150 power supply units is supplied, as a form of pulsed electromagnetic field, to the first and second quantum energy generating coils 360a, 360b, which, then generate pulsed electromagnetic fields in directions, opposing each other, but both perpendicular to the direction of the current, the fields overlapping and dissipating each other, thereby creating quantum energy in a state of zero strength electromagnetic filed.

The reference numeral 101 in FIG. 10 indicates a contact terminal as connected to an output conductive wire of the reverse polarity converter 200, which is connected to the output of any one unit of the power supply 100 selected from the group consisting of the first 110, second 120, third 130, fourth 140, and fifth 150 power supply units.

FIG. 11a is a sectional view of the wearable quantum treatment device integrated with a top 420, as shown in FIG. 8, FIG. 9a, FIG. 9b, FIG. 9c, FIG. 9d, FIG. 9e, embodied in the first 351, second 352, third 353, fourth 354, fifth 355, sixth 356 forms and a seventh form 357.

With reference to the drawings enclosed, the first and second quantum energy generating coils 360a, 360b integrated with such tops 420 covering the torso as a jumper, etc, may be made in any one selected from the group consisting of: weaving a garment with a conductive fabric, partially spraying a conductive material, through a perforated frame having a form of a quantum energy generating coil, onto a normal fabric; attaching the device to a fabric of a flexible printed circuit board having a form of a quantum energy generating coil; laser cutting a conductive metal sheet into a form of a quantum energy generating coil to insert or stack the same between an upper fabric and a lower fabric and then sewing them together; or winding a conductive metal wire or a sheathed electric wire over a base fabric to make a quantum energy generating coil, which is then stacked and sewed with a covering fabric.

And when the quantum energy generating coils 360a, 360b are integrated with such tops as a jumper, etc., a plurality of temperature sensors (not shown) for detecting a temperature of the body part (head) where the device is applied as well as the quantum energy generating coils 360a, 360b are also provided with intervals therebetween; the temperatures detected as above are provided, in real time, via a conductive wire (not shown), to a control unit of any one selected from the group consisting of: the first 110, second 120, third 130, fourth 140, fifth 150 power supply units, which, in turn, controls the output power of the selected power supply unit.

And the plurality of first and second quantum energy generating coils 360a, 360b integrated with a top 420 covering the torso are disposed in such areas of the head of a user as the forehead and the back or both sides of the head so as to make the two groups of the coils facing each other, and the windings of the two groups of coils are made in directions opposing each other so that power from any one of the power supply 100 selected from the group consisting of the first 110, second 120, third 130, fourth 140, fifth 150 power supply units is supplied, as a form of pulsed electromagnetic field, to the first and second quantum energy generating coils 360a 360b, which, then generate pulsed electromagnetic fields in directions, opposing each other, but both perpendicular to the direction of the current, the fields overlapping and dissipating each other, thereby creating quantum energy in a state of zero strength electromagnetic filed.

The reference numeral 101 in FIG. 11a indicates a contact terminal as connected to an output conductive wire of the reverse polarity converter 200, which is connected to the output of any one unit of the power supply 100 selected from the group consisting of the first 110, second 120, third 130, fourth 140, and fifth 150 power supply units.

FIG. 11b is a sectional view of the wearable quantum treatment device integrated with a pair of gloves for hands 430, as shown in FIG. 8, FIG. 9a, FIG. 9b, FIG. 9c, FIG. 9d, FIG. 9e, embodied in the first 351, second 352, third 353, fourth 354, fifth 355, sixth 356 forms and a seventh form 357. With reference to the drawings enclosed, the first and second quantum energy generating coils 360a, 360b integrated with a pair of gloves for hands 430, may be made in any one selected from the group consisting of: weaving a garment with a conductive fabric, partially spraying a conductive material, through a perforated frame having a form of a quantum energy generating coil, onto a normal fabric; attaching the device to a fabric of a flexible printed circuit board having a form of a quantum energy generating coil; laser cutting a conductive metal sheet into a form of a quantum energy generating coil to insert or stack the same between an upper fabric and a lower fabric and then sewing them together; or winding a conductive metal wire or a sheathed electric wire over a base fabric to make a quantum energy generating coil, which is then stacked and sewed up with a covering fabric.

And when the quantum energy generating coils 360a, 360b are integrated with a pair of gloves for hands 430, a plurality of temperature sensors (not shown) for detecting a temperature of the body part (head) where the device is applied as well as the quantum energy generating coils 360a, 360b are also provided with intervals therebetween; the temperatures detected as above are provided, in real time, via a conductive wire (not shown), to a control unit of any one selected from the group consisting of: the first 110, second 120, third 130, fourth 140, fifth 150 power supply units, which, in turn, controls the output power of the selected power supply unit.

And the plurality of first and second quantum energy generating coils 360a, 360b integrated with a pair of gloves for hands 430 are disposed at the front and the back of the hands so as to make the two groups of the coils facing each other, and the windings of the two groups of coils are made in directions opposing each other so that power from any one of the power supply 100 selected from the group consisting of the first 110, second 120, third 130, fourth 140, fifth 150 power supply units is supplied, as a form of pulsed electromagnetic field, to the first and second quantum energy generating coils 360a, 360b, which, then generate pulsed electromagnetic fields in directions, opposing each other, but both perpendicular to the direction of the current, the fields overlapping and dissipating each other, thereby creating quantum energy in a state of zero strength electromagnetic filed.

The reference numeral 101 in FIG. 11b indicates a contact terminal as connected to an output conductive wire of the reverse polarity converter 200, which is connected to the output of any one unit of the power supply 100 selected from the group consisting of the first 110, second 120, third 130, fourth 140, and fifth 150 power supply units.

FIG. 11c is a sectional view of the wearable quantum treatment device integrated with a pair of trousers for a lower body 440, as shown in FIG. 8, FIG. 9a, FIG. 9b, FIG. 9c, FIG. 9d, FIG. 9e, embodied in the first 351, second 352, third 353, fourth 354, fifth 355, sixth 356 forms and a seventh form 357. With reference to the drawings enclosed, the first and second quantum energy generating coils 360a, 360b integrated with a pair of trousers 440 for a lower body, may be made in any one selected from the group consisting of: weaving a garment with a conductive fabric, partially spraying a conductive material, through a perforated frame having a form of a quantum energy generating coil, onto a normal fabric; attaching the device to a fabric of a flexible printed circuit board having a form of a quantum energy generating coil; laser cutting a conductive metal sheet into a form of a quantum energy generating coil to insert or stack the same between an upper fabric and a lower fabric and then sewing them together; or winding a conductive metal wire or a sheathed electric wire over a base fabric to make a quantum energy generating coil, which is then stacked and sewed up with a covering fabric.

And when the quantum energy generating coils 360a, 360b are integrated with a pair of trousers 440, a plurality of temperature sensors (not shown) for detecting a temperature of the body part (head) where the device is applied as well as the quantum energy generating coils 360a, 360b are also provided with intervals therebetween; the temperatures detected as above are provided, in real time, via a conductive wire (not shown), to a control unit of any one selected from the group consisting of: the first 110, second 120, third 130, fourth 140, fifth 150 power supply units, which, in turn, controls the output power of the selected power supply unit.

And the plurality of first and second quantum energy generating coils 360a, 360b integrated with a pair of trousers 440 for a lower body are disposed in such areas at the front and back or both sides of the pair of trousers so as to make the two groups of the coils facing each other, and the windings of the two groups of coils are made in directions opposing each other so that power from any one of the power supply 100 selected from the group consisting of the first 110, second 120, third 130, fourth 140, fifth 150 power supply units is supplied, as a form of pulsed electromagnetic field, to the first and second quantum energy generating coils 360a, 360b, which, then generate pulsed electromagnetic fields in directions, opposing each other, but both perpendicular to the direction of the current, the fields overlapping and dissipating each other, thereby creating quantum energy in a state of zero strength electromagnetic filed.

The reference numeral 101 in FIG. 11c indicates a contact terminal as connected to an output conductive wire of the reverse polarity converter 200, which is connected to the output of any one unit of the power supply 100 selected from the group consisting of the first 110, second 120, third 130, fourth 140, and fifth 150 power supply units.

FIG. 11d is a sectional view of the wearable quantum treatment device integrated with a pair of socks or shoes (not shown) 450, as shown in FIG. 8, FIG. 9a, FIG. 9b, FIG. 9c, FIG. 9d, FIG. 9e, embodied in the first 351, second 352, third 353, fourth 354, fifth 355, sixth 356 forms and a seventh form 357 (not shown).

With reference to the drawings enclosed, the first and second quantum energy generating coils 360a, 360b integrated with a pair of socks 450 or shoes (not shown), may be made in any one selected from the group consisting of: weaving a garment with a conductive fabric, partially spraying a conductive material, through a perforated frame having a form of a quantum energy generating coil, onto a normal fabric; attaching the device to a fabric of a flexible printed circuit board having a form of a quantum energy generating coil; laser cutting a conductive metal sheet into a form of a quantum energy generating coil to insert or stack the same between an upper fabric and a lower fabric and then sewing them together; or winding a conductive metal wire or a sheathed electric wire over a base fabric to make a quantum energy generating coil, which is then stacked and sewed up with a covering fabric.

And when the quantum energy generating coils 360a, 360b are integrated with a pair of socks or shoes (not shown) 450, a plurality of temperature sensors (not shown) for detecting a temperature of the body part (feet) where the device is applied as well as the quantum energy generating coils 360a, 360b are also provided with intervals therebetween; the temperatures detected as above are provided, in real time, via a conductive wire (not shown), to a control unit of any one selected from the group consisting of: the first 110, second 120, third 130, fourth 140, fifth 150 power supply units, which, in turn, controls the output power of the selected power supply unit.

And the plurality of first and second quantum energy generating coils 360a, 360b integrated with a pair of socks or shoes (not shown) 450 are disposed in such areas as the top and bottom so as to make the two groups of the coils facing each other, and the windings of the two groups of coils are made in directions opposing each other so that power from any one of the power supply 100 selected from the group consisting of the first 110, second 120, third 130, fourth 140, fifth 150 power supply units is supplied, as a form of pulsed electromagnetic field, to the first and second quantum energy generating coils 360a, 360b, which, then generate pulsed electromagnetic fields in directions, opposing to each other, but both perpendicular to the direction of the current, the fields overlapping and dissipating each other, thereby creating quantum energy in a state of zero strength electromagnetic filed.

The reference numeral 101 in FIG. 11d indicates a contact terminal as connected to an output conductive wire of the reverse polarity converter 200, which is connected to the output of any one unit of the power supply 100 selected from the group consisting of the first 110, second 120, third 130, fourth 140, and fifth 150 power supply units.

With any one power supply unit with a control function integrated, supplying a variable power to the first and second quantum energy generating coils 360a, 360b, selected from the group consisting of the first 110, second 120, third 130, fourth 140, fifth 150 power supply units, an quantum resonance spectrometer applied an electromagnetic wave corresponding to that of an organ selected as a target of analysis to the body of a user of the present disclosure lying on a test table to detect and analyze a weak electromagnetic energy, as resonating with the electromagnetic wave, of the user so as to determine whether the bio-data of the user thus obtained indicates any abnormalities, more particularly, determines an amount of quantum energy to be irradiated enough to recover or treat an abnormal state based on a comparison of a resonance value of an organ of the body under a predetermined level with a reference value of electromagnetic resonance or electromagnetic energy for a healthy state.

The first and second quantum energy generating coils 360a, 360b supply a variable power, as a form of pulsed electromagnetic field, satisfying such parameters as variable current and voltage, variable frequency, strength of electromagnetic field, and power supply time, etc., to the reverse polarity converter 200, which, in turn, based on the information programmed and stored, in advance, in the control circuit, reverses the polarity of its output and supplied power as a form of pulsed electromagnetic field, via the contact terminal 101, to the quantum energy generating coils 360a, 360b integrated with a hat 410 covering the head, a top 440 for the torso, a pair of gloves 430 for hands, a bottom 440 for the lower body, or a pair of socks or shoes (not shown) 450, embodied in any one selected from the group consisting of the first to sixth forms and a seventh form as a combination thereof and with the first and second coils wound in direction opposing each other, but both perpendicular to the direction of the current so as to generate pulsed electromagnetic fields in directions, opposing each other, but both perpendicular to the direction of the current, the fields overlapping and dissipating each other, thereby creating quantum energy in a state of zero strength electromagnetic field such that the first coils 360a as disposed at the front of the body irradiate quantum energy toward the spine, while the second coils 360b as disposed at the back of the body irradiate quantum energy toward the front of the body, thereby the two electromagnetic fields thus created overlapping and dissipating each other to eventually create quantum energy in a state of zero strength electromagnetic field, which is irradiated to the inside of the body, that is, the entities of molecules, cells, tissues, and organs constituting the head, torso, hands, lower body, and feet, whereby those entities constituting body parts, when their resonance is below a normal level, are restored to their normal level; the water molecules of the body part where the electromagnetic fields are irradiated will have electrostatic attractive force, thereby reducing the hydrogen bonds between electric dipoles while increasing the level of coherence as well as decreasing the size of water clusters leading to a “micro-cluster” phenomenon in a permanently stabilized ground state; a weak electric disturbance is caused in the body water leading to polarization, thereby normalizing the quantum waves of the body water distorted by such conditions as diseases or abnormalities; a selected power supply unit supplies a power to the first and second quantum energy generating coils 360a, 360b, which, in turn, as the first and second coils are disposed in directions opposing each other, generate electromagnetic fields in such directions, but both perpendicular to the direction of the current, whereby the two electromagnetic fields overlap and dissipate each other, thereby creating quantum energy in a state of zero strength electromagnetic field, which is irradiated to such entities inside the body as molecules, cells, tissues, or organs, whereby such minerals as sodium and calcium get more active; vortexes turning in a helical way are induced in those helical structures or chakras as cell membranes, collagen, DNA, thereby improving health; the heat emitted from the first and second quantum energy generating coils 360a, 360b increases the body temperature more than a normal level (36.5° C.), which is beneficial to improving the activity of cells, the immune system, and blood cells as well as blood circulation, thereby improving health.

And examples of improving health and treating disease will be described in detail, but the applications of the present disclosure should not be construed as limited to those examples, but including other applications.

The function of quantum wave field Robert Becker, an orthopedist in the U.S. suggests in his research report on the meridian system and acupuncture points that those skin areas where acupuncture points are located have lower impedance (electrical impedance is the measure of the opposition that a circuit presents to a current when a voltage is applied) than their neighboring areas, and thus concludes that “Qi” in oriental medicine is a weak energy (quantum wave fields), which is closely related to transferring information inside a body.

And he further found out that when a narrow light beam is directed to acupuncture points, light beam is radiated from their neighboring areas of the skin. This research suggests that “Qi” is “a weak energy” (quantum wave field), which functions as a messenger of information through the meridian system.

The extracellular matrix as filling space between cells is a three dimensional network comprising collagen shaped as double or triple helix, thus having excellent piezoelectric properties. Besides, other numerous proteins including DNA having a helical structure in a body are piezoelectric, thereby suggesting that they have quantum wave fields and a function of transmitting information.

It has been reported that every cell not only has its own quantum wave field but also radiates its quantum wave field; thus a sick cell sends its quantum wave field signal to other cells, thereby making those other cells sick as well. This is an adverse effect of a quantum wave field.

As such, a quantum wave field has a function of transmitting information; thus exchange of information in a body may be carried out not only by neuronal firing and hormones but also via quantum wave fields.

### Function of Carrier Wave

A German scientist Fritz-Albert Popp suggests that the most important one of the functions of biophotons is the one as carrier wave. When modulating the amplitude and frequency of a high frequency wave, thereby transferring information between molecules, cells, tissues, or organs in a body, a carrier wave is required, which is carried out by biophotons; therefore, without biophotons, cells, tissues and organs cannot survive. That is, without information transferred by biophotons, any chemical reactions cannot happen.

### Recognition of External Quantum Wave Fields

Quantum wave fields inside a body are capable of recognizing external quantum wave fields; more particularly, when a quantum wave field of a frequency band identical to that of a quantum wave field inside a body is irradiated onto various body tissues, some frequencies act beneficially to the body, but some other frequencies do not depending on their frequency bands, which is observed at a level of such entities as molecules or cells, which means the capacity to recognize external quantum wave fields varies depending on levels of such entities as molecule, cells or organs.

It has been reported that when exposed to an external quantum wave field, a body recognizes the field via a harmonic oscillator inside. The harmonic oscillator herein is based on the idea that a frequency can resonate with another frequency if they are in relationship of a positive integer multiple. That is, a frequency of 2 Hz is different from a frequency of 12 Hz, but they can resonate with each other because 12 is one of the positive integer multiples of 2. Atoms and molecules inside a body have their own unique quantum wave fields; they can recognize, or resonate with, external quantum wave fields due to harmonics thereof even if their frequencies are different.

It has been reported that when a body is exposed to external quantum wave fields, the quantum wave fields of sodium or calcium ions inside the body first recognize and then amplify those external wave fields to change them into strong energy. That is, sodium or calcium ions are those which recognize external quantum wave fields. To elaborate on the amplification of quantum wave fields, it has bee reported that there are many helical structures such as cell membranes, collagens, DNA, chakras, etc., which induce, according to their helical structures, vortices, which contributes to the great amplification of energy. It is just like a cyclotron.

### Absorption of Energy by Zero Point Field of Space

The quantum wave fields inside a body work for absorbing energy of zero point fields in space, as summarized as below.
It has been reported that ions inside a body have their unique quantum wave fields, which help in introducing zero point energy of space, by rotating it perpendicularly into a three-dimensional space inside the body. According to the report, the zero point energy does not affect the body at all while randomly passing in the three dimensional space. However, it has been suggested that under a special condition that the structure of space is bent at right angle the zero point energy can be introduced inside the three dimensional space of our body. That is, the ions inside a body bend the structures of quantum wave fields at a right angle, thereby introducing zero point energy of space into the body.
It has been also reported that when being low in their quantum wave fields, cells can absorb zero point energy from the outer space to stabilize the quantum energy thereof. The phenomenon of absorbing zero point energy from space is called “breaking symmetry”; absorbing zero point energy of space is determined by the quantum wave fields of cells. “Breaking symmetry” herein means a phenomenon in which zero point energy of a four-dimensional flowing like a longitudinal wave bends at a right angle to change into a three-dimensional electromagnetic field as a transverse wave. That is, it is a phenomenon of changing the dimension of an energy field.
It has been reported that tissues in a body comprise a variety of liquid crystal structures, which function for absorbing zero point energy of space. And the liquid crystal structures not only absorb zero point energy of space but also convert the same into electromagnetic energy. That is, zero point energy of space is “information energy”, so to cause physiological changes in a body, it has to be converted into “force generating energy”, i.e., electromagnetic energy, which requires special structures for converting zero point energy into electromagnetic energy, which are found, as liquid crystal form, in brain and many other areas in a body.
   When calcium ions in a body go through cell membranes, they use special ion channels. The ion channels have a helical structure, so when ions go through these channels, they move in a helical form; thus, for example, it has been reported that if the resonant frequency of the helical movement of calcium ions is correctly calculated, the results are just like those obtained from a cyclotron. So it is suggested that when in helical movements, calcium ions absorb zero point energy of space.
It has been reported that chemicals dissolved in the water of a body help the quantum wave fields formed between water molecules and the chemicals absorb zero point energy of space.
DNA and RNA, reportedly, exist in a body as crystal form, so their crystalline structures form quantum wave fields, which absorb zero point energy of space. So DNA and RNA are called “Life Crystal”.

### Function of Self-Organization

Quantum wave fields are, reportedly, capable of self-organization, which has been most studied by Ilya Prigogine, the Nobel laureate in chemistry in 1977. In summary, he suggested, on self-organization, that when the seemingly disorderly molecules continue to receive external energy, they continue to be excited, and while breaking the equilibrium reach a critical point, when new orderly structures are created. He called this new structures “dissipative structures”, which, he suggested, is the place where the self-organization capabilities arise.

He further stated that the dissipative structures are characterized in that they are open systems which can accept materials or energy from outside; show non-linear reactions; are capable of not only negative but also positive feedback; and are capable of creating order from disorder.

He claimed that self-organization is a common and natural phenomenon observed in nature, and corroborated his idea with the examples as below.
When mirrors are installed both ends of a light emitting tube like neon signs and then voltage is applied thereonto, the gas atoms are excited to a high level energy and then drop to a lower level, thereby emitting light. While lights of various frequency bands are radiated and moving around between mirrors, they continue to excite other gas atoms and return them back to a ground state. During this process, only the light of the frequency band corresponding to the distance between mirrors is maintained, and the rest of them offset each other. The light of a constant and coherent wave spontaneously generated is a laser light. This is a kind of self-organization phenomenon which happens when the quantum wave fields of photons are strengthened.
15 varieties of enzymes are collected and energy is continuously applied thereonto for enough time, so as to excite the enzymes and induce them into a system far from equilibrium. As a result, a peculiar phenomenon happens where enzymes create enzymes. Enzymes, as commonly known, facilitate reactions of other chemicals, so it is so peculiar when they generate themselves. This peculiar behavior of enzymes is nothing but a result of self-organization caused by strengthening of quantum wave fields.
In chemistry, there is a phenomenon called “Rayleigh-Bénard convection”. When slowly heating a large pot of a flat bottom filled with water, due to the conductive heat, the water portion at the bottom rises while the water portion at the top goes down, and then the flow of water converts into convection when the difference between the bottom and the top reaches a critical point, whereby a great number of molecules are transported in a stable movement, thus forming hexagonal shapes like a honeycomb, a new order of molecules. These Bénard cells are a result of self-organization caused by strengthening of quantum wave fields of molecules.
In chemistry, there is a phenomenon called “a Belousov-Zhabotinsky reaction”. First, such materials as cerium sulfate, malonic acid, potassium bromide, etc. are added to a sulfuric acid solution, which then is slowly heated to induce chemical reactions. At the initial stage when the amount of heat applied to the solution is low, that is, at a state of equilibrium, the solution maintains its normal state without peculiarities. Slowly increasing the temperature breaks the equilibrium, so the solution reaches a critical point. Then suddenly, a dramatic event happens when depending on which mixture is more generated by autocatalysts, the solution changes its color in an order of blue, purple, and red, and then reverses the order and then repeats the cycle. For this phenomenon, may be made an analogy that you put 1 million of white table tennis balls and 1 million of black table tennis balls into a tank with an window, and then, while shaking the tank, may observe that the balls are colliding each while disorderly mixed, and then suddenly you may see the color of the balls change entirely into white, and then into black, and then repeat the cycle at an interval. This is a Belousov-Zhabotinsky reaction. This phenomenon is a self-organization which is caused when quantum wave fields are strengthened.

### Storage and Memory of Information

It has been known that quantum wave fields can store and memorize information. Below are the facts proved in this field.
When special solute molecules are added to water, the water molecules cover the solute molecules, which is called hydration. It has been reported that hydration intensifies quantum wave fields of water as well as imparts memory to water.
When lysozyme, a lytic enzyme for bacteria, is diluted with water of intensified quantum wave fields, it may be, reportedly, observed that no matter how much diluted, the water can memorize the enzyme; thus the physiological reactions of the enzyme can be seen in the water.
It has been also reported that when water of intensified quantum wave fields is irradiated by external electromagnetic fields, the water can memorize the information of the electromagnetic fields. So it is possible that a patient can be treated with water of the quantum wave fields which have been intensified with ultraviolet ray, visible ray, x-ray or electromagnetic fields. Especially when irradiated with a frequency of 10 KHz band, water can, reportedly, better memorize information with its intensified quantum wave fields.
It has been also reported that water in a bottle may be exposed less than one minute to electromagnetic fields coming from solenoid coils, thereby intensifying the quantum wave fields of the water as well as improving its memory.
A report suggests that after intensifying quantum wave fields of some water and rendering the water memorize some electromagnetic fields of a special frequency, the water can be used for various purposes. For example, the water of intensified quantum wave fields charged with a particular frequency of less than 500 Hz and a range of giga hertz can be used for removing water scales in industrial boilers; the water memorizing a particular frequency can be used for facilitating the growth of plants. Conversely, the water memorizing eletrostatic fields, direct current electromagnetic fields, supersonic waves can suppress the growth of plants. Just as if, when storing some information in a hard disk, you need to first format the disk, safely storing some information in water requires a kind of formatting the water with electromagnetic fields.

### DESCRIPTIONS OF THE SYMBOLS

| | |
|---|---|
| 101: Adhesive terminal | |
| 110: First power supply unit | 111: Battery cell |
| 112: DC/DC converter | 113: Control unit |
| 113a: Voltage determining unit | 113b: Voltage and current |
| | detecting sensors |
| 113c: Control circuit | |
| 120: Second power supply unit | 121: Step-down transformer |
| 122: Rectifier circuit | 123: Control unit |
| 123a: Input module | 123b: Operation module |
| 123c: Control module | 144: Current detecting sensor |
| 130: Third power supply unit | 131: AC power generating unit |
| 131a: First electrode | 131b: Piezoelectric material |
| 131c: Second electrode | 131d: Conductive wire |
| 132: AC/DC converter | 133: DC/DC converter |
| 134: Control module | 134a: Current detecting sensor |
| 134b: Variable controller | 134c: First controller |
| 134d: Second controller | |
| 140: Fourth power supply unit | 141: Rectifier |
| 142: Converter | 143: Inverter |
| 144: Resonant reactor | 145: Pulse transformer |
| 146: Control unit | 147: Gate actuating unit |
| 148a: First condenser | 148b: Second condenser |
| 150: Fifth power supply unit | 151: Power supply unit |
| 151a: AC power supply unit | 151b: DC power supply unit |
| 152: AC/DC converter | 153: Automatic Transfer |
| | Switch (AST) |
| 154: Low frequency outputting unit | 154a: Oscillator |
| 154b: Frequency allocation unit | 154c: Control unit |
| 154d: Amplifier | 155: Switching device |
| 156: Control unit | |
| 200: Reverse polarity converter | 201: Positive pole wire |
| 202: Negative pole wire | 211: First switch |
| 211a: Contact 1 | 211b: Contact 2 |
| 212: Second switch | 212a: Contact 1 |
| 212b: Contact 2 | 212c: Contact 3 |
| 212d: Contact 4 | 212a-1: Input wire 1 |
| 212b-1: Input wire 2 | 212c-1: Input wire 3 |
| 212d-1: Input wire 4 | |
| 212a-2: Output wire 1 | 212b-2: Output wire 2 |
| 212c-2: Output wire 3 | 212d-2: Output wire 4 |
| 311: Porous fabric structure | 312: Pores of porous fabric |
| | structure |
| 313: Conductive material layer | 314: Resin layer |
| 321: Fabric | 322: Working table |
| 323: Frame | 324: Sprayer |
| 331: Base film | 332: Resin composition |
| 332a: First resin | 332b: Second resin |
| 332c: Inorganic material | 332d: Hardening media |
| 341: Fabric | 342: Metal wire |
| 343: Stacking quantum coils on fabric | 344: Sewing quantum coils with |
| | fabric |
| 351: Lower base fabric | 352: Metal wire or sheathed line |
| 353: Upper base fabric | 354: Stacking quantum coils on |
| | fabric |
| 355: Sewing fabric just like winding | |
| quantum coils | |
| 360a: First quantum energy generating | |
| coils | |
| 360b: Second quantum energy | |
| generating coils | |
| 351: First form of the first and second | |
| quantum energy generating coils | |
| 352: Second form of the first and | |
| second quantum energy generating coils | |
| 353: Third form of the first and second | |
| quantum energy generating coils | |
| 354: Fourth form of the first and second | |
| quantum energy generating coils | |
| 355: Fifth form of the first and second | |
| quantum energy generating coils | |
| 356: Sixth form of the first and second | |
| quantum energy generating coils | |
| 410: Hat | |
| 420: Top | |
| 430: Hands | |
| 440: Pair of trousers | |
| 450: Pair of socks and shoes | |

1. A wearable quantum treatment device, comprising: a power supply unit selected from a group consisting of:
a first power supply unit having a battery cell, a DC/DC converter, a voltage determining unit, a voltage and current detecting sensor, a control circuit, a power cut-off switch, and a control unit integrated with a temperature sensor detecting surface temperature of first and second quantum energy generating coils and temperature of a user's body part where the wearable quantum treatment device is applied, configured to supply a variable power as a form of pulsed electromagnetic field to the first and second quantum energy generating coils;
a second power supply unit having a step-down transformer, a rectifier circuit, an input module, an operation module, and a control module, a control unit integrated with a temperature sensor detecting surface temperature of the first and second quantum energy generating coils and temperature of a user's body part where the wearable quantum treatment device is applied, configured to supply a variable power as a form of pulsed electromagnetic field to the first and second quantum energy generating coils;
a third power supply unit having an AC power generating unit with a first electrode, a piezoelectric material, a second electrode, and a conductive wire integrated, an AC/DC/converter, a DC/DC converter, and a control unit with a current detecting unit, a variable controller, a first controller, a second controller, a temperature sensor detecting surface temperature of the first and second quantum energy generating coils and temperature of a user's body part where the wearable quantum treatment device is applied, configured to supply a variable power as a form of pulsed electromagnetic field to the first and second quantum energy generating coils;
a fourth power supply unit having a rectifier, a converter, an inverter, a resonant reactor, a pulse transformer, a control unit with a temperature sensor detecting surface temperature of the first and second quantum energy generating coils and temperature of a users body part where the wearable quantum treatment device is applied, a gate actuating unit, a first condenser, a second condenser, configured to supply a variable power as a form of pulsed electromagnetic field to the first and second quantum energy generating coils;
a fifth power supply unit having a power supply portion being either of an AC power supply portion and a DC power supply portion, an AC/DC converter, an Automatic Transfer Switch, a low frequency generating and outputting unit with an oscillator, a frequency allocation unit, a controller, an amplifier integrated, a switching device, a control unit with a temperature sensor detecting surface temperature of the first and second quantum energy generating coils and temperature of a users body part where the wearable quantum treatment device is applied, configured to supply a variable power as a form of pulsed electromagnetic field to the first and second quantum energy generating coils;
a reverse polarity converter, wherein a variable (current or voltage) DC power from a power supply unit selected from a group of power supply units consisting of the first power supply unit, the second power supply unit, the third power supply unit, the fourth power supply unit, and the fifth power supply unit, is provided first, via a positive conductive wire and a negative conductive wire, to a first uncontrollable switch and input wires of a second switch controlled by a micro controller, by a control of which a contact 3 of the second switch comes into contact with a contact 2, and a contact 1 comes into contact with a contact 4, thereby reversing the polarities (that is, a negative becomes a positive, and vice versa), whereby a variable power is applied to the first and second quantum energy generating coils via output wires of the second switch;
the quantum energy generating coils embodied in a configuration selected from a group consisting of:
a first material configuration, made by weaving conductive fibers, having a porous fabric structure, pores of the porous fabric structure, a conductive material layer filling the pores, a resin layer coating an outer surface of the conductive material layer;
a second material configuration made by first spreading a fabric of a porous fabric structure on a working table, and then arranging a frame perforated in a form of a quantum energy generating coil selected from a first form to a 6th form or a seventh 7th form as combination of the first to the 6th forms, in contact with the fabric prepared, and then spraying, with a sprayer prepared, a conductive material thereonto, thereby filling the conductive material layer into the pores of the porous fabric structure and then drying the same at a temperature ranging from 80 to 120° C. for 1 to 2 hours;
a third material configuration made by a process comprising: preparing a base film; supplying a first resin to a reactor; supplying a second resin; providing an inorganic filler; adding and stirring a hardening medium, thereby producing a resin composition; spreading the resin composition; stacking quantum coils; hot-forming; hardening, thereby producing a flexible printed circuit board;
a fourth material configuration made by laser-cutting a metal disc made of any one of a group of materials consisting of copper (Cu), stainless steel (ST304, STS316L), silver (Ag), titanium (Ti) into one selected from a group consisting of the first form to the sixth form and a seventh form as a combination of the first to sixth forms to make the quantum energy generating coils, which are then disposed on and in contact with a porous base fabric prepared in advance, and are covered by, and in contact with, a covering fabric, and eventually sewed up together;
a fifth material configuration by using quantum coils of a wire made of any one of the group of materials consisting of copper, nickel, etc., or a sheathed wire; and then disposing the quantum coils, wound in any one of the first form to the sixth form or a seventh form as a combination of the first form to the sixth form, in contact with a base fabric prepared in advance, onto which a covering fabric separately prepared is stacked and sewed up together to finish the assembly of quantum energy generating coils; embodied in a form selected from a group consisting of a first form in which a coil is wound from top to bottom, with intervals, forming a zigzag pattern; a second form in which a coil is wound, laterally, with intervals, forming a zigzag pattern; a third form which is a solenoid coil transformed into a shape of disc; a fourth form of toroid coil; a fifth form of Möbius coil; a sixth form of Helmholtz coil; a seventh form of a combination of the above forms; integrated with a head covering part of a cap, a hat, an earflap hat or a helmet, etc; on such tops as a jacket covering from the neck to the hip joint, a pique shirt, a shirt with an adjustable neck, a suit, a jumper, etc.; on a pair of gloves for hands; on such bottoms covering from the waist to the ankle as a pair of thermal underwear bottoms, a pair of trousers, a skirt, etc.; on a pair of socks for feet.

2. The wearable quantum treatment device of claim 1, wherein the first power supply unit of the power supply comprises:
a battery cell;
a DC/DC converter; and
a control unit with such control capabilities as a voltage determining unit, a voltage and current detecting sensor, a control circuit, a power cut-off switch integrated, configured to supply a variable power as a form of pulsed electromagnetic field to the first and second quantum energy generating coils.

3. The wearable quantum treatment device of claim 1, wherein the second power supply unit of the power supply comprises:
a step-down transformer;
a rectifier circuit;
a control unit with such control capabilities as an input module, an operating module, a control module integrated;
a temperature sensor detecting surface temperature of the quantum energy generating coils as well as surface temperature of a user's body part (head, torso, hands, lower body, or feet) where the wearable quantum treatment device is applied; and
a current detecting sensor, thereby supplying a variable electric power as a form of pulsed electromagnetic field to the first and second quantum energy generating coils.

4. The wearable quantum treatment device of claim 1, wherein the third power supply unit of the power supply comprises:
an AC power generating unit with a first electrode, a piezoelectric material, a second electrode, and a conductive wire integrated; and
an AC/DC converter, a DC/DC converter, a control unit with such control capabilities as a current detecting unit, a variable controller, a first controller, a second controller, a temperature sensor detecting surface temperature of the quantum energy generating coils as well as surface temperature of a user's body part (head, torso, hands, lower body, or feet) where the wearable quantum treatment device is applied, thereby supplying a variable electric power as a form of pulsed electromagnetic field to the first and second quantum energy generating coils.

5. The wearable quantum treatment device of claim 1, wherein the fourth power supply unit of the power supply comprises:
a rectifier;
a converter;
an inverter;
a resonant reactor;
a pulse transformer;
a control unit with such control capabilities as a temperature sensor detecting surface temperature of the quantum energy generating coils as well as surface temperature of a user's body part (head, torso, hands, lower body, or feet) where the wearable quantum treatment device is applied;
a gate actuating unit;
a first condenser; and
a second condenser, thereby supplying a variable electric power as a form of a pulsed electromagnetic field to the first and second quantum energy generating coils.

6. The wearable quantum treatment device of claim 1, wherein the fifth power supply unit of the power supply comprises:
a power supply portion being either of an AC power supply portion and a DC power supply portion;
an AC/DC converter;
an Automatic Transfer Switch;
a low frequency generating and outputting unit with an oscillator, a frequency allocation unit, a controller, an amplifier integrated; a switching device; and
a control unit with a temperature sensor detecting surface temperature of the first and second quantum energy generating coils and temperature of a user's body part (head, torso, hands, lower body, or feet) where the wearable quantum treatment device is applied, configured to supply a variable power as a form of pulsed electromagnetic field to the first and second quantum energy generating coils.

7. The wearable quantum treatment device of claim 1,
further comprising a reverse polarity converter, wherein a variable (current or voltage) DC power from a power supply unit of a power supply selected from a group consisting of a first power supply unit, a second power supply unit, a third power supply unit, a fourth power supply unit, a fifth power supply unit, is provided first, via a positive conductive wire and a negative conductive wire, to a first uncontrollable switch and input wires of a second switch controlled by a micro controller, by control of which a contact 3 of the second switch comes into contact with a contact 2, and a contact 1 comes into contact with a contact 4, thereby reversing the polarities (that is, a negative becomes a positive, and vice versa), whereby a variable power is applied to the first and second quantum energy generating coils via output wires of the second switch.

8. The wearable quantum treatment device of claim 1, wherein:
a first material configuration of the quantum energy generating coils includes:
a porous fabric structure having pores,
a conductive material (layer) filling the pores, and
a resin layer coating the conductive material layer; and
the first material configuration is produced by steps including:
selecting a porous fabric structure made of one from a group consisting of nylon, polyamide, polyaramid, polyetherimide, polyacrylonitrile, poly aniline, polyethyleneoxide, polyethylene naphthalate, polybutylene terephthalate, styrene butadiene rubber, polystyrene, polyvinyl chloride, polyvinyl alcohol, polyvinylidene, fluoride, polyvinyl butylene, polyurethane, polybenzoxazole, polybenzimidazole, polyamide imide, polyethyleneterephthalate, polyethylene, polypropylene, polycarbonate, copolymers and mixtures thereof;
preparing, for filling the pores of the porous fabric structure thus prepared, a mixture of one selected from a group consisting of such metals as iron (Fe), zirconium (Zr), beryllium (Be), manganese (Mn), germanium (Ge), zinc (Zn), palladium (Pd), copper (Cu), rhodium (Rh), magnesium (Mg), tin (Sn), silver (Au), bismuth (Bi), aluminum (Al), lithium (Li), iridium (Jr), ruthenium (Ru), platinum (Pt), titanium (Ti), osmium (Os), gallium (Ga), rubidium (Rb), indium (In), and metal nano-wires, conductive polymers, with a metal content being 30 to 70% relative to a total amount of the conductive material, and, for a purpose of imparting heat emitting properties, one selected from a group consisting of carbon nanotube (CNT), graphene, carbon black in an amount of 30 to 70% relative to a total amount of the conductive material;
stacking a frame, as perforated in a form selected from a group of the first to sixth forms for the first and second quantum energy generating coils and a seventh form as a combination of the first and sixth forms, onto and in contact with the fabric of the porous fabric structure prepared on a working table;
spraying the mixture prepared as above onto the frame to form a filling layer;
putting, into a reactor with a mixer, a material, for forming a resin layer, selected from a group consisting of silicon oxide, silicon nitrile, aluminum oxide, acrylic resin, epoxy resin, silicon resin, fluoropolymer, styrene resin, polyolefin resin, thermoplastic elastomer, polyoxy alkylene resin, polyester resin, polyvinyl chloride resin, polycarbonate resin, polyphenylene sulfide resin, polyamide resin, polysilicon epoxy, and mixtures thereof;
adding a solvent selected from a group consisting of oxane solvent, amine solvent, polyol solvent, alkane solvent, benzene solvent, ether solvent in an appropriate amount;
mixing the composition thus prepared in the reactor with a mixer to adjust viscosity thereof;
spraying the composition partially onto a form selected from the group consisting of the first and sixth forms for the first and second quantum energy generating coils, and a seventh form in an entire area of a garment (a hat, a top, a pair of gloves, a bottom, a pair of socks), so as to form a resin layer for coating; and
drying and cooling down the resin layer thus prepared by a dryer for 1 to 2 hours at a temperature of 80 to 120° C., thereby eventually producing a fabric with conductive and optionally heat emitting properties.

9. The wearable quantum treatment device of claim 1, wherein a second material configuration of the quantum energy generating coils is produced by steps of:
spreading a fabric of a porous fabric structure on a working table;
stacking, onto and in contact with the fabric thus prepared, a frame, as perforated in a form selected from the group of the first to sixth forms for the first and second quantum energy generating coils and a seventh form as a combination of the first and sixth forms;
preparing, for filling the pores of the porous fabric structure thus prepared, a mixture of one selected from a group consisting of such metals as iron (Fe), zirconium (Zr), beryllium (Be), manganese (Mn), germanium (Ge), zinc (Zn), palladium (Pd), copper (Cu), rhodium (Rh), magnesium (Mg), tin (Sn), silver (Au), bismuth (Bi), aluminum (Al), lithium (Li), iridium (Ir), ruthenium (Ru), platinum (Pt), titanium (Ti), osmium (Os), gallium (Ga), rubidium (Rb), indium (In), and metal nano-wires, conductive polymers, with a metal content being 30 to 70% relative to a total amount of the conductive material, and, for a purpose of imparting heat emitting properties, one selected from the group consisting of carbon nanotube (CNT), graphene, carbon black in an amount of 30 to 70% relative to a total amount of the conductive material by mixing the composition in a reactor to adjust viscosity thereof;
spraying the mixture prepared as above onto the frame to form a filling layer;
preparing a material, for forming a resin layer 321c, selected from the group consisting of silicon oxide, silicon nitrile, aluminum oxide, acrylic resin, epoxy resin, silicon resin, fluoropolymer, styrene resin, polyolefin resin, thermoplastic elastomer, polyoxy alkylene resin, polyester resin, polyvinyl chloride resin, polycarbonate resin, polyphenylene sulfide resin, polyamide resin, polysilicon epoxy, and mixtures thereof;
spraying, after adjusting viscosity, the composition partially onto a form selected from a group consisting of the first and sixth forms for the first and second quantum energy generating coils, and a seventh form in an entire area of a garment (a hat, a top, a pair of gloves, a bottom, a pair of socks), so as to form a resin layer for coating; and
drying and cooling down the resin layer thus prepared by a dryer for 1 to 2 hours at a temperature of 80 to 120° C., thereby eventually producing a fabric with conductive and optionally heat emitting properties.

10. The wearable quantum treatment device of claim 1, wherein a third material configuration of the quantum energy generating coils is produced by a method comprising:
preparing a base film made of one selected from a group consisting polyimide, PET (polyethylene terephthalate), PMMA (polymethyl methacrylate), PDMS (polymethylsiloxane), polyester film, polyethylene film, polypropylene film;
putting, for making a resin composition, into a reactor one selected from a group consisting of tetrahydrophthalic anhydride, hexahydrophthalic anhydride, methyltetrahydrophthalic anhydride, PDMA(pylomellitic dianhydride), BTDA(benzophenon teyracarboxylic dianhydride), TMEG(ethylene glycol bis-trimellitic dianhydride), TMTA(glycerol tris-trimellitic dianhydride), MCTC(5-(2,5-dioxotetrahydropryle)-3-cyclohexene-1,2-dicarboxylic anhydride), TDA(2,5-dioxotetranhydrofuran-3-yl)1,2,3,4-tetrahydronaphthalene-1,2-dicarboxyl anhydride);
adding a filler material selected from a group consisting of such conductive materials as carbon black (C), acetylene black (C), Ketjenblack (C) in an appropriate amount (30 to 90%); any one of the group of nitride filler, alumina, silica as well as a hardening agent in an amount of 30 to 70% relative to a total amount of inorganic materials;
mixing the composition thus made by a mixer for a predetermined time so as to achieve a predetermined level of viscosity;
spraying the resin composition prepared as above with a predetermined thickness, by using a spray gun onto a base film;
selecting any one material selected from the group consisting of copper (Cu), silver (Ag), gold (Au), etc.;
machining the selected material into any one of the group consisting of the first, second, third, fourth, fifth, sixth forms and a seventh form as a combination thereof for the first and second quantum energy generating coils;
disposing the machined material in contact with a base film with the resin composition spread thereon; and
compressing, by a hot-forming machine, the stack, thereby producing the first and second quantum energy generating coils with conductive and heat generating properties.

11. The wearable quantum treatment device of claim 1, wherein a fourth material configuration of the quantum energy generating coils is produced by a method comprising steps of:
first preparing a porous base fabric;
selecting any one material from a group consisting of copper (Cu), stainless steel (STS304, STS316L), silver (Ag), titanium (Ti), etc. as in a form of disc or carbon sheet molded with a high compression;
machining;
laser-cutting the selected material into any one form selected from a group consisting of the first to sixth forms and a seventh form as a combination thereof for the quantum energy generating coils;
disposing the machined coils on, and in contact with, the porous base fabric;
stacking a covering fabric on the quantum energy generating coils; and
sewing up, by a sewing machine, the stack just like winding the coils, thereby eventually producing a fabric of a predetermined area having conductive and optionally heat emitting properties.

12. The wearable quantum treatment device of claim 1, wherein a fifth material configuration of the quantum energy generating coils is produced by a method comprising:
preparing a base fabric of a predetermined area;
selecting any one from a group of materials conductive and heat emitting with a diameter of 50 μm to 2 mm, consisting of silver, copper, nickel, carbon fiber, graphite fiber, medium temperature carbonized fiber, activated carbon fiber;
winding the material covered with an insulating material or a sheathed heat emitting material in any one selected from the group consisting of the first to sixth forms and a seventh form as a combination thereof for the first and second quantum energy generating coils;
disposing the first and second quantum energy generating coils thus wound on, and in contact with, the base fabric;
stacking a covering fabric thereon;
sewing, by using a sewing machine, the stack to fix the quantum energy generating coils;
cutting, by using a cutter, the stack into a plurality of quantum energy generating coils of a predetermined area; and
sewing the stack thus cut with such garments as a head cover, a top, a pair of grousers, or a pair of socks, thereby eventually producing a fabric having conductive and optionally heat emitting properties.

13. The wearable quantum treatment device of claim 1, wherein the first and second quantum energy generating coils are embodied in any one selected from a group consisting of:
a first form wherein a coil is wound from top to bottom, with intervals, forming a zigzag pattern; the overall pattern has fine creases to prevent damages to the coils from distortions caused by the extensions or bending of the body;
a second form wherein a coil is wound, laterally, with intervals, forming a zigzag pattern; the overall pattern has fine creases to prevent damages to the coils from distortions caused by the extensions or bending of the body;
a third form wherein a solenoid coil is transformed into a shape of disc, wherein the overall pattern has fine creases to prevent damages to the coils from distortions caused by the extensions or bending of the body with concentric circles of coils wound from the center outwardly or from the circumference inwardly;
a fourth form wherein a coil is wound in a toroidal shape, wherein the overall pattern has fine creases to prevent damages to the coils from distortions caused by the extensions or bending of the body with the coils wound in a shape of helix with winding spacings;
a fifth form wherein a coil is wound in a Möbius shape, wherein the overall pattern has fine creases to prevent damages to the coils from distortions caused by the extensions or bending of the body in a shape of double helix like a number “8”;
a sixth form wherein a coil is wound in a shape of Helmholtz coil, wherein the overall pattern has fine creases to prevent damages to the coils from distortions caused by the extensions or bending of the body with a shape of double circle; and
a seventh form as a combination of the first to sixth forms.

14. The wearable quantum treatment device of claim 1, wherein a method for producing and integrating the first and second quantum energy generating coils may be selected from a group consisting of:
weaving a porous fabric of conductive and heat emitting properties embodied in any one or more selected from a group consisting of the first to the seventh forms for the first and second quantum energy generating coils;
sewing with, or attaching to, a garment, the first and second quantum energy generating coils embodied in a form of flexible printed circuit board with conductive and heat emitting properties;
inserting, stacking, and sewing up together, the first and second quantum energy generating coils, made by laser-cutting a conductive metal sheet or a carbon sheet of conductive and heat emitting properties molded with a high compression into any one or more selected from the group consisting of the first to the seventh forms, between upper and lower base covering fabrics;
winding, stacking, and sewing up, by a sewing machine, the first and second quantum energy generating coils made of a wire of silver, copper or nickel, etc., covered with an insulating material with a diameter of 50 μm to 2 mm or a wire of a sheathed heat emitting material (carbon fiber, nichrome wire), embodied in any one or more selected from the group consisting of the first and seventh forms, between upper and lower base covering fabrics, and then attaching the first and second quantum energy generating coils thus produced to one of such garments as a hat, a top, a pair of gloves, a bottom, a pair of socks or shoes.

15. The wearable quantum treatment device of claim 1, wherein the wearable quantum treatment device may be applied to such different categorized areas as a head, a torso including both arms, both hands, a lower body including hips and both legs, feet including socks and shoes areas such that to an upper portion or one side of a lower portion or an upper portion is attached an adhesive terminal for providing a power from an output (positive, negative poles) of the reverse polarity converter, which received power from any one selected from a group consisting the first power supply unit, the second power supply unit, the third power supply unit, the fourth power supply unit, and the fifth power supply unit, to the first and second quantum energy generating coils.

16. The wearable quantum treatment device of claim 1, wherein one or more methods for integrating the first and second quantum energy generating coils with such garments as a hat, a top, a pair of gloves, and a bottom, and a pair of socks or shoes may be selected from a group consisting of:
weaving the first and second quantum energy generating coils directly with a conductive and heat emitting material;
partially spraying a conductive and heat emitting material onto a common porous fabric;
making or sewing the first and second quantum energy generating coils with a conductive and heat emitting flexible printed circuit board;
inserting, stacking, and sewing together, the first and second quantum energy generating coils as a form of metal sheet of conductive and heat emitting properties between upper and lower fabrics; and
disposing the first and second quantum energy generating coils made of a conductive and heat emitting metal wire coated with an insulating material or a sheathed heat emitting material (carbon fiber, nichrome wire) on, and in contact with, a base fabric, and then further providing, stacking, and sewing up together, a covering fabric.

17. The wearable quantum treatment device of claim 1, wherein:
the first quantum energy generating coils are disposed at a front (of a body) of such garments as a hat, a top, a pair of gloves, a bottom, a pair of socks or shoes whereas the second quantum energy generating coils are disposed at a back (of a body) of said garments such that winding of the first quantum energy generating coils is made in a direction opposite to that of the second quantum energy generating coils; and
any one power supply unit of the power supply, selected from the group consisting of the first, the second, the third, the fourth, and the fifth power supply units, supplies a variable (current or voltage) power as controlled by a controller at its output to a reverse polarity converter, which in turn provides power to the first and second quantum energy generating coils wound in directions opposing each other, whereby two variable pulsed electromagnetic fields are generated in directions perpendicular to that of current and opposing each other, thereby overlapping and dissipating each other, thus eventually irradiating quantum energy in a state of zero strength electromagnetic field to a user's body.

18. The wearable quantum treatment device of claim 1, wherein based on biometric information of a user's body, analyzed, calculated and input with an input module and a quantum resonance spectrometer of a control unit, that is, based on comparison of resonance values of target organs in a user's body with a reference value, each of the first, second, third, fourth, and fifth power supply units of the power supply supplies a variable power of a pulsed electromagnetic field, which meets such parameters as variable voltage, variable current, variable frequency, and strength of electromagnetic field, power supply time as corresponding to resonance values and strength of electromagnetic fields required to recover a healthy state of the user, to the first and second quantum energy generating coils.

19. The wearable quantum treatment device of claim 1, wherein:
any one selected from a group consisting of the first, second, third, fourth, and fifth power supply units of the power supply supplies power to the first and second quantum energy generating coils embodied in one selected from the group consisting of the first to the seventh forms and disposed at the front or back or both sides of such garments as a hat covering a head, a top for a torso, a pair of gloves for hands, a bottom for a lower body, or a pair of socks or shoes for feet, such that as the windings of the two sets of coils are opposite to each other, an electromagnetic field generated, in a direction perpendicular to that of current, by the first quantum energy generating coils disposed at a front of a user's body is irradiated toward a spine, whereas an electromagnetic field generated by the second quantum energy generating coils disposed at a back of the user's body is irradiated toward the front of the user's body, whereby two pulsed electromagnetic fields generated in directions opposite to each other overlap and dissipate each other at the center of the user's body, thus quantum energy irradiated to such entities constituting the user's body parts (the head, torso, hands, lower body, and feet) as molecules, cells, tissues, and organs, whereby those entities having resonance values lower than a reference level are restored to a healthy state;
the water molecules of the body part where the electromagnetic fields are irradiated will have electrostatic attractive force, thereby reducing the hydrogen bonds between electric dipoles while increasing the level of coherence as well as decreasing the size of water clusters leading to a “micro-cluster” phenomenon in a permanently stabilized ground state;
a weak electric disturbance is caused in the body water leading to polarization, thereby normalizing the quantum waves of the body water distorted by such conditions as diseases or abnormalities;
the two electromagnetic fields overlap and dissipate each other, thereby creating quantum energy in a state of zero strength electromagnetic field, which is irradiated to such entities inside the body as molecules, cells, tissues, or organs, whereby such minerals as sodium and calcium get more active;
vortexes turning in a helical way are induced in those helical structures or chakras as cell membranes, collagen, DNA, thereby improving health; and
the heat emitted from the first and second quantum energy generating coils increases the body temperature more than a normal level (36.5° C.), which is beneficial to improving the activity of cells, the immune system, and blood cells as well as blood circulation, thereby improving health.

20. The wearable quantum treatment device of claim 1, wherein:
any one selected from the group consisting of the first, second, third, fourth, and fifth power supply units of the power supply, which provides power to the first and second quantum energy generating coils disposed in various garment areas, comprise an input module having such functions as timer and switching device, which contribute to decreasing power consumption, thereby helping the battery last longer; especially with the switching devices, power may be supplied, or cut off from, individually or in a sequential way, to a plurality of the first and second quantum energy generating coils disposed in such areas of garments as a head cover, a top including both arms and both hands, a bottom including both legs and hips, a pair of socks or shoes, whereby along with a pounding effect, pulsed electromagnetic fields generated in directions opposite to each other overlap and dissipate each other, thus creating and irradiating quantum energy in a state of zero strength electromagnetic field to a user's body.
